(19) United States
(12) Patent Application Publication
(10) Pub. No.: US 2025/0384957 A1
(43) Pub. Date: Dec. 18, 2025

(54) SYSTEM AND METHOD FOR CLASSIFYING CANCER AND CLASSIFYING BENIGN AND MALIGNANT NEOPLASM

(71) Applicant: THE HOSPITAL FOR SICK CHILDREN, Toronto (CA)

(72) Inventors: Adam SHLIEN, Toronto (CA); Federico COMITANI, Toronto (CA)

(21) Appl. No.: 18/837,073

(22) PCT Filed: Feb. 10, 2023
(86) PCT No.: PCT/CA2023/050175

§ 371 Date: Aug. 8, 2024

Related U.S. Application Data
(60) Provisional application No. 63/309,255, filed on Feb. 11, 2022.
(60) Provisional application No. 63/350,947, filed on Jun. 10, 2022.

(51) Int. Cl. G16B 25/10; G06N 3/0464; G16B 20/00; G16B 30/00; G16B 40/20; G16H 50/20

(52) U.S. Cl. CPC G16B 25/10; G06N 3/0464; G16B 20/00; G16B 30/00; G16B 40/20; G16H 50/20

(57) ABSTRACT

The present invention relates to systems and methods for classification of cancer from gene expression input data. The method including: receiving a training dataset including nucleic acid data points from one or more samples; identifying classes in the training dataset including performing recursive clustering by, at each successive iteration, performing a search to identify clusters based on similarity, wherein each class of the classes is associated with a tumor; training a machine learning model to associate the nucleic acid data points of the training dataset with the identified classes; receiving the gene expression input data for classification; classifying, using the trained machine learning model, the gene expression input data as one or more of the identified classes; and outputting the classification of the gene expression input data.

### TECHNICAL FIELD

The present invention relates to tools for cancer diagnosis; and more particularly, to a system and method for classifying cancer and classifying benign and malignant neoplasm.

### BACKGROUND

Cancer is a major cause of disease-related death with an estimated 9.6 million people having died from various forms of cancer in 2017. Additionally, cancer is an especially prevalent cause of disease-related death in children with over 400,000 cases per year. Childhood cancers proliferate rapidly. Fast and accurate diagnosis is therefore critical; however, it is frequently delayed, incorrect, or even missed entirely for children with cancer. This is due, in part, to differences between adult and childhood tumors, which are less common, can emerge from embryonic tissue, and impact different cell types. Most adult extracranial solid tumors are carcinomas, while neoplasms of mesodermal and embryonal origin are more frequent in children. One third of childhood cancer are leukemias, but they are not as commonly found in adults. The same is true for neuroblastomas, a highly heterogeneous cancer that ranges from a spontaneously regressing form in infants to a malignant progressing entity in older children and adolescents, yet is rarely found in adults.

A small number of childhood cancers harbor pathognomonic fusions that can be used as diagnostic markers. Cancer genome sequencing can detect these fusions but, beyond these markers, has only limited diagnostic utility in childhood cancer. Genome sequencing can reveal the history of the tumor, including the mutations preceding its malignant transformation, but these are not necessarily reflective of the tumor's ongoing expression program or the active pathology of the disease. There is currently no comprehensive molecular assay that can aid in the diagnosis of all pediatric cancers.

It is therefore an object of the present invention to provide a system and method in which the above disadvantages are obviated or mitigated, and attainment of various desirable attributes is facilitated.

### SUMMARY

In an aspect, there is provided a computer-implemented method for classification of cancer from gene expression input data, the method comprising: receiving a training dataset comprising nucleic acid data points from one or more samples; identifying classes in the training dataset comprising performing recursive clustering by, at each successive iteration, performing a search to identify clusters based on similarity, wherein each class of the classes is associated with a tumor; training a machine learning model to associate the nucleic acid data points of the training dataset with the identified classes; receiving the gene expression input data for classification; classifying, using the trained machine learning model, the gene expression input data as one or more of the identified classes; and outputting the classification of the gene expression input data.

In a particular case of the method, the method further comprises, prior to identifying classes, performing removal of low information features.

In another case of the method, removal of low information features comprises ranking features by their respective variance and removing features whose variance is below a pre-determined cut-off.

In yet another case of the method, the variance is determined using Shannon's entropy.

In yet another case of the method, the method further comprises, prior to identifying the classes, performing non-linear dimensionality reduction.

In yet another case of the method, non-linear dimensionality reduction comprises performing Uniform Manifold Approximation and Projection.

In yet another case of the method, optimizing the input dataset comprises determining a score representative of a quality of the identified clusters by determining a ratio between cohesion of the clusters and separation of the clusters.

In yet another case of the method, the method further comprises performing a grid search to determine optimized parameters for identifying the clusters.

In yet another case of the method, determining the optimized parameters is repeated for each iteration using only data included in a given cluster to identify hierarchical subclusters.

In yet another case of the method, once clusters and subclusters are identified, each cluster is successively selected and parameters are optimized internally for such cluster, further iterations of optimization are performed following a branch stemming from such cluster.

In yet another case of the method, performing recursive clustering comprises: iteratively identifying clusters with different parameters for each iteration; evaluating each identification of clusters using an internal validation score; and selecting the identification of clusters with the highest score.

In yet another case of the method, the method further comprises determining complexity of a hierarchical branch stemming from each cluster based on a number of offspring hierarchical nodes.

In yet another case of the method, the trained machine learning model outputs membership probability to one or more of the identified classes.

In yet another case of the method, the trained machine learning model comprises an ensemble of convolutional neural networks.

In yet another case of the method, each convolutional neural network comprises one or more one-dimensional convolutional layers, followed by one or more fully connected layers with dropout, and followed by a fully connected layer.

In yet another case of the method, the trained machine learning model is multiclass such that the gene expression input data can be assigned to more than one class.

In yet another case of the method, the method further comprises performing agglomerative clustering on log 2 normalized expressions of the gene expression input data prior to classification.

In yet another case of the method, the input dataset comprises genes as features and expression counts of such genes as values.

In yet another case of the method, the reference dataset comprises gene expression from healthy tissue.

In yet another case of the method, the method further comprises identifying gene expression outliers in the gene expression input data prior to classification, comprising comparing gene expression for the input data against gene expression distributions of reference tumours and normal tissues.

In yet another case of the method, each of the classes are associated with a type of sarcoma tumor.

In yet another case of the method, the classes are associated with one or more of osteosarcoma, leiomyosarcoma, fusion-positive rhabdomyosarcoma, fusion-negative rhabdomyosarcoma, synovial sarcoma, and Ewing sarcoma.

In another aspect, there is provided a system for classification of cancer from gene expression input data, the system comprising: an input module to receive a training dataset comprising nucleic acid data points from one or more samples and receive the gene expression input data for classification; an optimization module to identify classes in the training dataset comprising performing recursive clustering by, at each successive iteration, performing a search to identify clusters based on similarity, wherein each class of the classes is associated with a tumor; a training module to train a machine learning algorithm to associate the nucleic acid datapoints of the training dataset with the identified classes; a classification module to classify, using the trained machine learning model, the gene expression input data as one or more of the identified classes; and an output module to output the classification of the gene expression input data.

In a particular case of the system, the optimization module, prior to identifying classes, performs removal of low information features.

In another case of the system, the removal of low information features comprises ranking features by their respective variance and removing features whose variance is below a pre-determined cut-off.

In yet another case of the system, the variance is determined using Shannon's entropy.

In yet another case of the system, the optimization module, prior to identifying classes, performs non-linear dimensionality reduction.

In yet another case of the system, the non-linear dimensionality reduction comprises performing Uniform Manifold Approximation and Projection.

In yet another case of the system, optimizing the input dataset comprises determining a score representative of a quality of the identified clusters by determining a ratio between cohesion of the clusters and separation of the clusters.

In yet another case of the system, the optimization module performs a grid search to determine optimized parameters for identifying the clusters.

In yet another case of the system, determining the optimized parameters is repeated for each iteration using only data included in a given cluster to identify hierarchical subclusters.

In yet another case of the system, once clusters and subclusters are identified, each cluster is successively selected and parameters are optimized internally for such cluster, further iterations of optimization are performed following a branch stemming from such cluster.

In yet another case of the system, performing recursive clustering comprises: iteratively identifying clusters with different parameters for each iteration; evaluating each identification of clusters using an internal validation score; and selecting the identification of clusters with the highest score.

In yet another case of the system, the optimization module further determines complexity of a hierarchical branch stemming from each cluster based on a number of offspring hierarchical nodes.

In yet another case of the system, the trained machine learning model outputs membership probability to one or more of the identified classes.

In yet another case of the system, the trained machine learning model comprises an ensemble of convolutional neural networks.

In yet another case of the system, each convolutional neural network comprises one or more one-dimensional convolutional layers, followed by one or more fully connected layers with dropout, and followed by a fully connected layer.

In yet another case of the system, the trained machine learning model is multiclass such that the gene expression input data can be assigned to more than one class.

In yet another case of the system, the classification module further performs agglomerative clustering on log 2 normalized expressions of the gene expression input data prior to classification.

In yet another case of the system, the reference dataset comprises gene expression from healthy tissue.

In yet another case of the system, the system further comprises an outlier module to identify gene expression outliers in the gene expression input data prior to classification, comprising comparing gene expression for the input data against gene expression distributions of reference tumours and normal tissues.

In yet another case of the system, each of the classes are associated with a type of sarcoma tumor.

In yet another case of the system, the classes are associated with one or more of osteosarcoma, leiomyosarcoma, fusion-positive rhabdomyosarcoma, fusion-negative rhabdomyosarcoma, synovial sarcoma, and Ewing sarcoma.

In yet another aspect, there is provided a computer-implemented method for classifying benign and malignant neoplasm from gene expression input data, the method comprising: receiving a training dataset comprising nucleic acid data points comprising a whole, or a substantial portion of, a transcriptome; identifying classes in the training dataset comprising performing recursive clustering by, at each successive iteration, performing a search to identify clusters based on similarity; training a machine learning model to associate the nucleic acid data points of the training dataset with the identified classes; classifying the gene expression input data as either benign or malignant neoplasm using the trained machine learning model; outputting the classifications of benign or malignant.

In a particular case of the method, the machine learning model comprises a Gradient Boosting ensemble, the Gradient Boosting ensemble comprises a set of classifiers and the learned weights of the classifiers are used to confirm expression pathways relevant to benign and malignant classifications.

In another case of the method, outputting the classifications of benign or malignant comprises outputting feature importance scores extracted from the classifications of the clusters.

In yet another case of the method, classifying the clusters as either benign or malignant neoplasm using the trained machine learning model further comprises extracting immune activity, interleukin and integrin signaling, and markers of thyroid carcinoma based on the classification.

In yet another case of the method, the method further comprises classifying the clustered data as either healthy or tumour-adjacent normal tissues using a second machine learning model, the second machine learning model comprising a Gradient

Boosting ensemble, and outputting the classification as either healthy or tumour-adjacent normal tissues.

In yet another case of the method, classifying the clustered data as either healthy or tumour-adjacent normal tissues using the second machine learning model further comprises extracting RAS and ESR1 pathways, cell adhesion, and locomotion and metabolic pathways based on the classification.

In yet another case of the method, the method further comprises classifying the classified benign or malignant neoplasm as either BRAF-like and non-BRAF-like using a third machine learning model, the third machine learning model comprising a Gradient Boosting ensemble, and outputting the classification as either BRAF-like and non-BRAF-like.

In yet another case of the method, classifying the classified benign or malignant neoplasm as either BRAF-like and non-BRAF-like using the third machine learning model further comprises extracting mammalian target of rapamycin (mTOR), GTPase and cell cycle based on the classification.

In yet another case of the method, the input dataset comprises nucleic acid data from healthy samples, and wherein the healthy samples are also classified as either BRAF-like and non-BRAF-like.

In yet another case of the method, the input dataset comprises nucleic acid data from thyroid samples.

In yet another aspect, there is provided a system for classification of benign and malignant neoplasm from gene expression input data, the system comprising one or more processors in communication with data storage to execute: an input module to receive a training dataset comprising nucleic acid data points comprising a whole, or a substantial portion of, a transcriptome; an optimization module to identify classes in the training dataset comprising performing recursive clustering by, at each successive iteration, performing a search to identify clusters based on similarity; a training module to train a machine learning model comprising a Gradient Boosting ensemble to associate the nucleic acid data points of the training dataset with the identified classes; a classification module to classify the gene expression input data as either benign or malignant neoplasm using the trained machine learning model; and an output module to output the classifications of benign or malignant.

In a particular case of the system, the machine learning model comprising a Gradient Boosting ensemble, the Gradient Boosting ensemble comprises a set of classifiers and the learned weights of the classifiers are used to confirm expression pathways relevant to benign and malignant classifications.

In another case of the system, outputting the classifications of benign or malignant comprises outputting feature importance scores extracted from the classifications of the clusters.

In yet another case of the system, classifying the clusters as either benign or malignant neoplasm using the trained machine learning model further comprises extracting immune activity, interleukin and integrin signaling, and markers of thyroid carcinoma based on the classification.

In yet another case of the system, the classification module further classifies the clustered data as either healthy or tumour-adjacent normal tissues using a second machine learning model, the second machine learning model comprising a Gradient Boosting ensemble, and outputting the classification as either healthy or tumour-adjacent normal tissues.

In yet another case of the system, classifying the clustered data as either healthy or tumour-adjacent normal tissues using the second machine learning model further comprises extracting RAS and ESR1 pathways, cell adhesion, and locomotion and metabolic pathways based on the classification.

In yet another case of the system, the classification module further classifies the classified benign or malignant neoplasm as either BRAF-like and non-BRAF-like using a third machine learning model, the third machine learning model comprising a Gradient Boosting ensemble, and outputting the classification as either BRAF-like and non-BRAF-like.

In yet another case of the system, classifying the classified benign or malignant neoplasm as either BRAF-like and non-BRAF-like using the third machine learning model further comprises extracting mammalian target of rapamycin (mTOR), GTPase and cell cycle based on the classification.

In yet another case of the system, the input dataset comprises nucleic acid data from healthy samples, and wherein the healthy samples are also classified as either BRAF-like and non-BRAF-like.

In yet another case of the system, the input dataset comprises nucleic acid data from thyroid samples.

In yet another aspect, there is provided a system for classification of a tumor from gene expression input data, comprising: (a) a clustering module; and (b) a classification module; wherein the clustering module performs one or more operations comprising generating a hierarchy of labels, wherein a label of the hierarchy of labels is associated with a tumor; wherein the classification module performs one or more operations comprising: training a classification model to associate gene expression training data with one or more labels of the hierarchy of labels; and associating the gene expression input data with one or more labels of the hierarchy of labels by processing the gene expression input data with the trained classification model.

In a particular case of the system, the gene expression input data comprises RNA sequence data.

In another case of the system, the RNA sequence data comprises one or more of polypeptide encoding sequence data and pseudogene sequence data.

In yet another case of the system, the RNA sequence data comprises sequence data encoding or corresponding with more than 18,000 polypeptides and/or pseudogenes.

In yet another case of the system, polypeptide encoding sequence data comprises sequence data encoding a plurality of genes and corresponding gene expression counts.

In yet another case of the system, generating the ground truth data comprises using agglomerative clustering on log 2 normalized expressions of the gene expression input data prior to classification.

In yet another case of the system, generating the ground truth data comprises iteratively clustering the gene expression input data, wherein during an iteration a score is calculated for a set of clusters.

In yet another case of the system, generating the ground truth data further comprises selecting a set of clusters with a highest score.

In yet another case of the system, the score is based at least in part on a ratio between cohesion of the clusters and separation of the clusters.

In yet another case of the system, iteratively clustering the gene expression data is performed using a grid search or tree-based method.

In yet another case of the system, the hierarchy comprises at least a first level and a second level.

In yet another case of the system, a label corresponding to a first class of tumor is associated with the first level and a label corresponding to a second class of tumor is associated with the second level, wherein the first class comprises the second class.

In yet another case of the system, the first class of tumor corresponds to a cluster of the gene expression input data, wherein the cluster groups a plurality of gene expression data items based at least in part on their similarity.

In yet another case of the system, the second class of tumor corresponds to a subset of the cluster, wherein the subcluster groups a plurality of gene expression data items of the cluster based at least in part on their similarity.

In yet another case of the system, the classification model is a multiclass model.

In yet another case of the system, the classification model is an ensemble model.

In yet another case of the system, the ensemble model comprises a plurality of neural networks.

In yet another case of the system, the neural networks are convolutional neural networks (CNNs).

In yet another case of the system, a convolutional neural network comprises one or more one-dimensional (1D) convolutional layers, one or more fully-connected layers with dropout, and a fully-connected layer.

In yet another case of the system, generating the ground truth data comprises removing low information features from the gene expression input data.

In yet another case of the system, removing low information features comprises (1) defining a threshold variance and (2) removing features of the gene expression input data with variances lower than the threshold variance.

In yet another case of the system, the variances are determined based at least in part using Shannon's entropy.

In yet another case of the system, generating the ground truth data comprises applying non-linear dimensionality reduction to the gene expression input data.

In yet another case of the system, the non-linear dimensionality reduction comprises Uniform Manifold Approximation and Projection (UMAP).

In yet another case of the system, matching the tumor with the one or more labels comprises generating a set of probabilities, a probability of the set of the probabilities indicating a quantitative degree of association between the tumor and at least one label of the one or more labels.

In yet another case of the system, the hierarchy of labels does not include any manual or human-generated labels.

In yet another aspect, there is provided a method for classification of a tumor from gene expression input data, performed using a system comprising a clustering module and a classification module, comprising: generating, by the clustering module, a hierarchy of labels, wherein a label of the hierarchy of labels is associated with a tumor; training, by the classification module, a classification model to associate gene expression training data with one or more labels of the hierarchy of labels; and associating, by the classification module, the gene expression input data with one or more labels of the hierarchy of labels by processing the gene expression input data with the trained classification model.

In another case of the method, the method further comprises determining or modifying a course of treatment based at least in part on the association of the gene expression input data with the one or more labels.

These and other aspects are contemplated and described herein. The foregoing summary sets out representative aspects of systems and methods to assist skilled readers in understanding the following detailed description.

### DESCRIPTION OF THE DRAWINGS

An embodiment of the present invention will now be described by way of example only with reference to the accompanying drawings, in which:

FIG. 1 is a block diagram showing a system for classification of cancer from gene expression input data using nucleic acid data points based clustering, according to an embodiment;

FIG. 2A is a flow chart showing a method for nucleic acid data points based clustering for classifying cancer, according to an embodiment;

FIG. 2B is a flow chart showing a method for classification of cancer using the nucleic acid data points based clustering, according to an embodiment;

FIG. 3 is an exemplary schematic representation of clustering and classification of RNASeq data, in accordance with the system of FIG. 1;

FIG. 4 is a graph showing classification performance as a function of the number of sequenced reads which can be quantified as hierarchical similarity between prediction probabilities obtained from subsampled data and an original sample;

FIG. 5 are graphs showing computation time for example experiments confirming the advantages of the system of FIG. 1;

FIG. 6 shows clusters hierarchy of the example experiments using 2-d UMAP projection of gene expression counts representative of a first level of hierarchy obtained with the system of FIG. 1 on a reference tumor dataset;

FIG. 7 shows a list of 27 transcriptional families identified in the example experiments at a first level of the hierarchy, where each class, number of members, total offspring subtypes, sex ratio, and age distribution are reported;

FIG. 8 shows a Population Weighted Splits (PaWS) scores for the example experiments measured for each of main 27 transcriptional families, where a marker size is proportional to a population of the cluster, and where the score quantifies a relative number of offspring for each class adjusted by the population bias;

FIG. 9 shows heterogeneity of the example experiments with a scatter plot showing median entropy and median absolute deviation for each normal and tumor subtype identified by multi-scale clustering, where marker size is proportional to population of the cluster, and where distributions of these values and their medians are shown for pediatric and adult samples, and where a Mann-Whitney U rank test p-values between these distributions are also shown showing pediatric malignancies have higher median and MAD entropy;

FIG. 10 shows intraclass entropy differences between pediatric (left) and adult (right) samples for the example experiments, where the pediatric samples show significantly higher entropy;

FIG. 11 shows per gene entropy distributions for each of 26 main tumor classes in the example experiments, where genes are grouped by their DeepLIFT importance score;

FIG. 12 shows, for the example experiments, normalized stemness (top of each class) and immune activity (bottom of each class) scores distributions for the full hierarchy of clusters within T002, mesodermal tumors with high immune infiltration, and T003, mesodermal tumors with high stemness;

FIG. 13 shows, for the example experiments, same samples projected onto a set of single-cell datapoints from embryonal bone tissue, showing how stem-high samples (T003) are more closely related to the embryonal tissue, and shows cosine distance of the centroids of mesodermal clusters from the embryonal bone tissue obtained from a 12-dimensional UMAP projection;

FIG. 14 shows BCOR- and CIC-mutated tumors identified in the example experiments using a 2-dimensional UMAP projection of CNS tumors by gene expression, a diagram representing the archetypical BCOR-ITD and BCOR-CCNB3 rearrangements, a BCOR expression distribution across representative CNS classes, showing a clear overexpression in BCOR-mutated samples (T031), an idiosyncratic transcriptional profile of BCOR mutations identified to show that it is sufficient to overcome the cell-of-origin attraction during the clustering process, a 2-dimensional UMAP projection of MESODERM STEM-high tumors by gene expression, a diagram representing the archetypical CIC-DUX4 fusion, an MYC expression distribution across representative mesodermal classes, showing a overexpression in T104, and, as for BCOR-mutated samples, the ratio of tumor types within T104 showing a mixture of sarcomas and CNS tumors;

FIG. 15 shows neuroblastoma and osteosarcoma tumors identified in the example experiments using a 2-dimensional UMAP projection of neuroblastoma subtypes by gene expression, overall survival curves for the neuroblastoma subtypes, showing significant prognostic stratification, distribution plots for the stemness (top of each class) and immune activity (bottom of each class) across neuroblastoma transcriptional subtypes, cell lineage distributions grouped by neuroblastoma subtype, normalized gene sets enrichment score of genes expressed downstream to MYCN amplification, across neuroblastoma subtypes and between samples in T064, a 2-dimensional UMAP projection of osteosarcoma subtypes, overall survival curves for the neuroblastoma subtypes, showing significant prognostic stratification, distributions of normalized gene sets enrichment scores describing cartilage and bone development across the identified osteosarcoma subtypes, and SP7 transcription factor expression distributions across osteosarcoma transcriptional subtypes;

FIG. 16 shows scores for hierarchy and pediatric classes for neuroblastoma in the example experiments showing excellent performance even for minor subtypes found deep in the hierarchy;

FIG. 17 shows class assignment and probability for neuroblastoma in the example experiments showing individual neuroblastomas expressed multiple transcriptional programs at the same time;

FIG. 18 shows class assignment probability and lineage for neuroblastoma in the example experiments showing uniqueness of subtype;

FIG. 19A is a flowchart illustrating a method for generating labels for use in classifying benign and malignant neoplasm, in accordance with an embodiment;

FIG. 19B is a flowchart illustrating a method for classifying benign and malignant neoplasm, in accordance with an embodiment;

FIG. 20 illustrates a summary of clinical information from the mixed-source dataset used in example experiments of the method of FIGS. 19A and 19B;

FIG. 21 illustrates a flowchart showing the main steps of the multi-scale iterative clustering approach, in accordance with the method of FIGS. 19A and 19B;

FIG. 22 illustrates two-dimensional UMAP representations of the full thyroid cohort, in accordance with the example experiments of FIG. 20;

FIG. 23 illustrates two-dimensional UMAP representations of the BRAF-like A subclass, in accordance with the example experiments of FIG. 20;

FIG. 24 illustrates two-dimensional UMAP representations of the RAS-like subclass, in accordance with the example experiments of FIG. 20;

FIG. 25 shows the first three layers of the thyroid samples cluster hierarchy, in accordance with the example experiments of FIG. 20;

FIG. 26 shows a heatmap illustrating the median normalized enrichment score of a curated subset of gene sets, in accordance with the example experiments of FIG. 20;

FIG. 27 shows UMAP projections of BRAF-like and RAS-like samples shaded as a function of their immune and invasiveness scores, in accordance with the example experiments of FIG. 20;

FIG. 28 shows distributions of the stemness, immune activity and invasiveness scores stratified by benign nodules or malignant, in accordance with the example experiments of FIG. 20;

FIG. 29 shows median normalized enrichment score for a set of representative gene sets across benign and malignant tumours, in accordance with the example experiments of FIG. 20;

FIG. 30 shows normalized enrichment score distributions for a group of in-house defined gene sets, in accordance with the example experiments of FIG. 20;

FIG. 31 shows median normalized enrichment score for a set of representative gene sets across healthy and tumour-adjacent normal samples, in accordance with the example experiments of FIG. 20;

FIG. 32 shows a confusion matrix of the multiclass GBoost classifier (left), healthy versus tumour-adjacent normals binary classifier (top right), and benign versus malignant tumour binary classifier (bottom right), in accordance with the example experiments of FIGS. 20; and

FIG. 33 shows performance scores for each of the classes predicted by the three GBoost models, in accordance with the example experiments of FIG. 20.

### DETAILED DESCRIPTION

Embodiments will now be described with reference to the figures. For simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Any module, unit, component, server, computer, computing device, mechanism, terminal or other device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Any application or module herein described may be implemented using computer readable/executable instructions that may be stored or otherwise held by such computer readable media and executed by the one or more processors.

### Introduction

RNA-sequencing has the potential to be a standalone “universal diagnostic assay” for cancer because it can differentiate and group tumors independently of their apparent genomic origin and because a critical number of tumor transcriptomes have been, or will soon be, sequenced. Most transcriptome-based tumor classifiers are built as fully supervised tools; they are reliant on the tumor's pre-existing label without allowing for much phenotypic variability. Yet, intra-tumoral transcriptional differences can be so pronounced that they result in both good and poor prognostic signatures within the same tumor. Stromal or immune cell infiltrates also add to this diversity and can confound existing tools.

In some embodiments of the present disclosure, advantageously, there is identification and use of features that uniquely define the gene expression profiles of childhood cancers compared to their adult counterparts. The present embodiments can be used to quantify the inter- and intra-type transcriptional variability of childhood cancer. By incorporating measures of transcriptional entropy, the heterogeneity of childhood cancer gene expression can be determined at multiple levels-both between subtly different tumor types as well as across major classes of cancer. Common belief was that childhood cancers are quiet because their genomes have very few mutations compared to adult cancers that have had many years to acquire mutations (and often results from external mutations causing agents, like smoking). Far from being “quiet”, the present inventors have determined that childhood tumors display more transcriptional diversity, both between and within tumor types, than most adult cancers. Thus, on a transcriptional level, childhood cancers are very noisy and maintain high transcriptional diversity. The present embodiments can make use of this variability and account for it, rather than neglect it, which can be leveraged to provide substantially improved diagnosis of childhood and adult cancer.

The present embodiments provide a scale-adaptive clustering approach (informally referred to as ‘RACCOON’—Robust Adaptive Coarse-to-fine Clusters OptimizatiON) for the unsupervised identification of tumor subtypes using RNA-Seq. The present embodiments also provide a classifier for childhood cancer (informally referred to as ‘OTTER’—Oncologic TranscripTome Expression Recognition), an ensemble of convolutional neural networks targeting this extensive hierarchy. Such embodiments are unique in scope, and perform robustly even when using a fraction of the RNA-Seq data (i.e., a few million reads).

There are a large number of types of cancer in both adults and children. For example, there are 114 recognized types of childhood cancer, and just as many rare entities that have not yet been given a name or which bear features spanning multiple diagnostic categories.

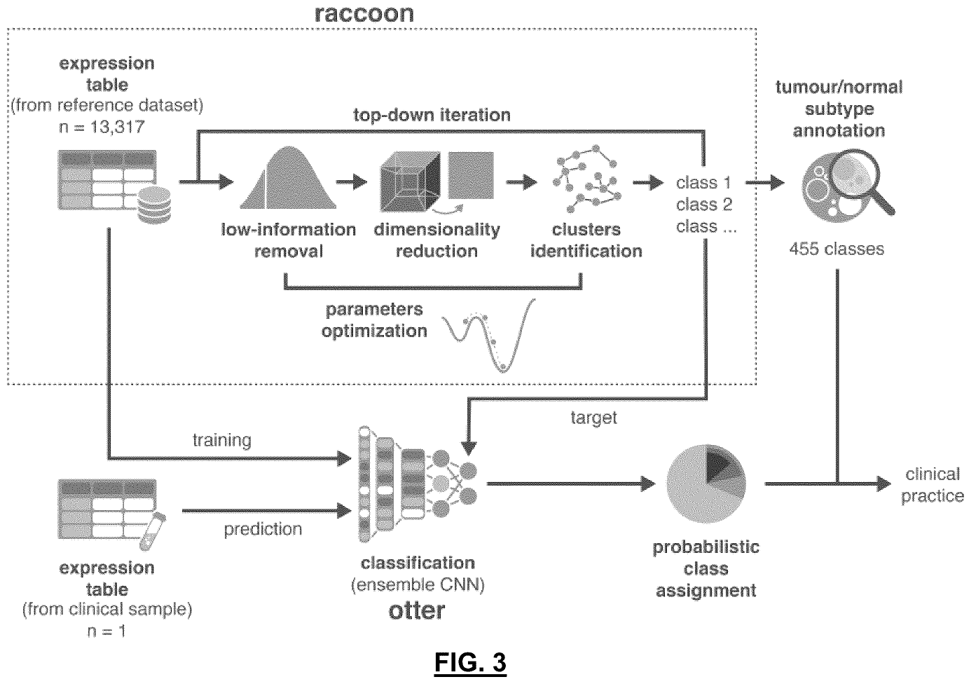

To develop a molecular definition of childhood cancer, the present embodiments provide an approach that reduces the complexity of RNA-sequenced tumors, then groups them into hierarchically organized clusters (as illustrated in the example of FIG. 3). This approach enables a deeper exploration of the transcriptional differences between and within tumor classes and facilitates the discovery of new tumor subtypes. The present embodiments have automatic optimization of parameters for low-information filtering, dimensionality reduction and clusters identification; thus, removing the need for tumor-type-specific expertise or intuition when choosing these critical parameters. Additionally, the present embodiments provide iterative top-down building of hierarchies of cancer in a way that is scale and dataset independent.

Additionally, the present embodiments provide a set of mono-dimensional convolutional neural networks (CNNs) to match individual patients undergoing active treatment to novel tumor classes with high accuracy. In some cases, these networks for tumor classification can be broad and shallow, suggesting that a large proportion of the transcriptome needs to be evaluated to accurately diagnose cancer but that there is only a limited complexity of interactions between the genes involved.

In an example, the top three performing CNNs can be integrated into an ensemble, which is referred to as OTTER. The ensemble of the present embodiments outperformed alternative transcriptome-based classifiers, while matching patients to a considerably larger set of 455 hierarchically structured classes across a pan-cancer atlas.

Because tumors can contain multiple distinct cell populations, as well as different components of stroma or immune infiltrate, in some cases, the classifier can be both multiclass and multilabel. As such, the output of the classifier can generate probabilities that a tumor belongs to a class, as well as its child classes, their children, and so on; thus, giving a highly refined view of a tumor's assigned subtype within a specific tumor “lineage.” With this approach, a patient's cancer can also match to multiple possibly unrelated clusters depending on, for example, the tumor's degree of differentiation and/or the admixture of distinct cell populations found within the same bulk tissue.

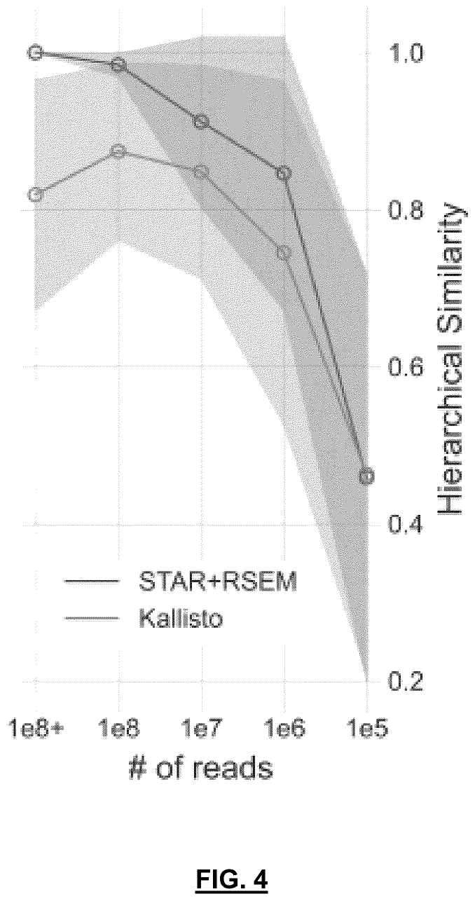

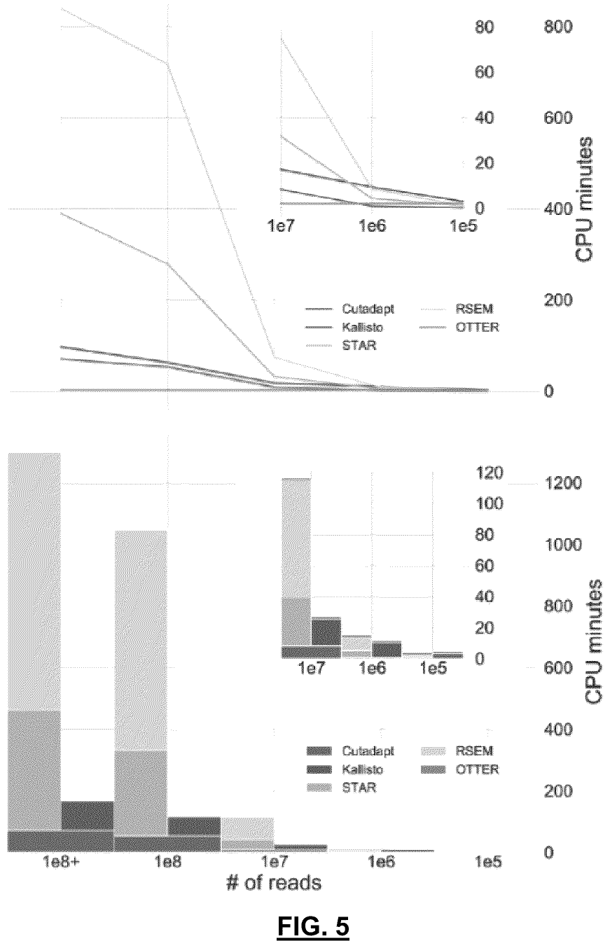

The example experiments confirmed that the ensemble maintains high performance across all cancer types, as well as in the presence of multiple tumor mixtures, high normal contamination, or technical noise. In the example experiments, the ensemble was trained on a full reference cohort, which includes samples at a range of sequencing depths. Surprisingly, the example experiments indicated that tumor matching is robust even with very shallow sequencing, as illustrated in the example of FIG. 4. Using only one million reads, the ensemble can output highly consistent predictions in just a few minutes (as illustrated in the example of FIG. 5). Thus, advantageously, the present embodiments provide, for example, a low cost approach for tumor diagnostics that maintains a high accuracy.

### System

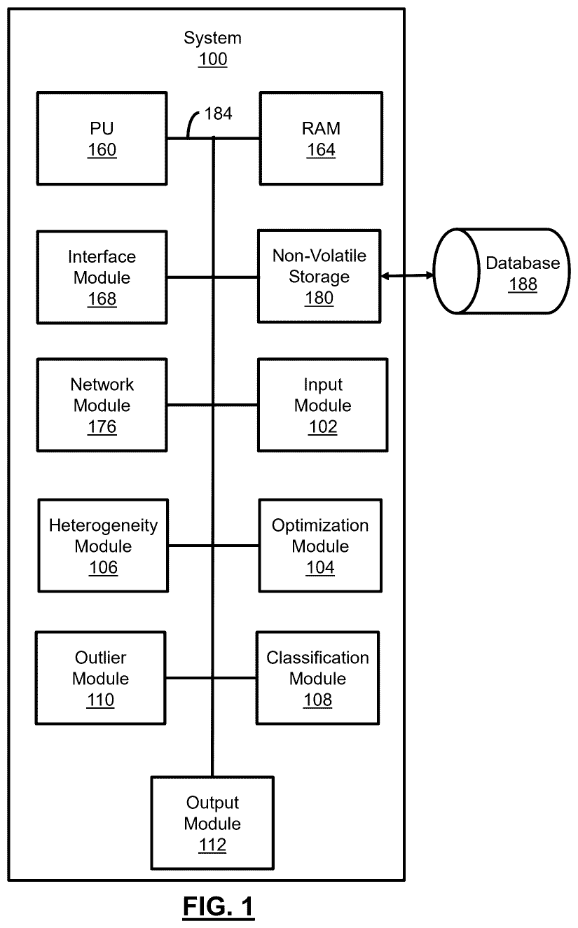

FIG. 1 illustrates a schematic diagram of a system 100 for classification of cancer from gene expression input data and for classifying benign and malignant neoplasm, according to various embodiments. As shown, the system 100 has a number of physical and logical components, including a processing unit (“PU”) 160, random access memory (“RAM”) 164, an interface module 168, a network module 176, non-volatile storage 180, and a local bus 184 enabling PU 160 to communicate with the other components. PU 160 can include one or more processors, a microprocessor, dedicated hardware, or other integrated processing circuits. RAM 264 provides relatively responsive volatile storage to PU 260. The interface module 268 enables input to be provided; for example, directly via a user input device, or indirectly, for example, via an external device. The interface module 168 also enables output to be provided; for example, directly via a user display, or indirectly, for example, sent over the network module 176. The network module 176 permits communication with other systems or computing devices; for example, over a local area network or over the Internet. Non-volatile storage 180 can store an operating system and programs, including computer-executable instructions for implementing the methods described herein, as well as any derivative or related data. In some cases, this data can be stored in a database 188. In some cases, during operation of the system 100, the operating system, the programs and the data may be retrieved from the non-volatile storage 280 and placed in RAM 264 to facilitate execution. In other embodiments, any operating system, programs, or instructions can be executed in hardware, specialized microprocessors, logic arrays, or the like. In some cases, the processing can be distributed over multiple computing devices.

In an embodiment, the PU 160 can be configured to execute a number of conceptual modules, for example, an input module 102, an optimization module 104, a heterogeneity module 106, a classification module 108, an outlier module 110, and an output module 112. In further cases, functions of the above modules can be combined or executed on other modules. In some cases, functions of the above modules can be executed on remote computing devices, such as centralized servers and cloud computing resources communicating over the network module 176.

Unsupervised clustering analyses may involve: (1) preprocessing, where feature selection is carried out to simplify the input space and shed noise, and normalization or standardization are applied if needed; (2) dimensionality reduction; and/or (3) clusters identification. In most cases, each of these steps may require some level of parametrization, which may be left to user experience and knowledge of the domain. Furthermore, when working with complex data, the relationship between datapoints (for example, the similarity in expression profiles among RNASeq samples) changes at different scales and clustering should take this into account.

While hierarchical clustering methods may rely on a fixed set of parameters and are thus susceptive to parametrization imprecisions. Additionally, they may not directly account for the change in relationship between samples at different scales, but rather may expect a single embedded space (that is a space that has been subjected to dimensionality reduction) to carry multi-scale information.

The system 100 advantageously addresses the above issues using an approach that is scale-adaptive and self-optimizing by working iteratively: for a given dataset, the optimization module 104 attempts to identify an optimal set of clusters, and then it repeats the identification internally for each of cluster separately. This is particularly advantageous when working with non-linear dimensionality reduction algorithms (for example, Uniform Manifold Approximation and Projection (UMAP)) because the resulting embedded space is strongly dependent on the data itself and similarity/distance relationships between and among data items. In an example, the input module 104 can receive a pan-cancer set of gene expression input data. Mapping performed by the optimization module 104 can prioritize separation of tumor types, but may lose information on subtypes at a finer grain. The dimensionality reduction can be repeated for each tumor type separately to be able to accurately identify subtypes (e.g. myeloid vs lymphoid leukemia, or, at a deeper level, leukemia subtypes defined by specific lesions). And each of these steps, a full re-parametrization can be performed since an optimal set of parameters at large scales may miss structures that are relevant at smaller scales and vice versa.

Advantageously, using the optimization module 104, the impact on user experience and intuition has been greatly reduced. The library can include an optimizer that automatically searches for the best set of parameters for each of the steps discussed above (preprocessing, dimensionality reduction and clusters identification). A score to quantify the quality of the clustering can be generated by comparing the relative distances of the datapoints across clusters in the embedded space can be used for the search.

### Method

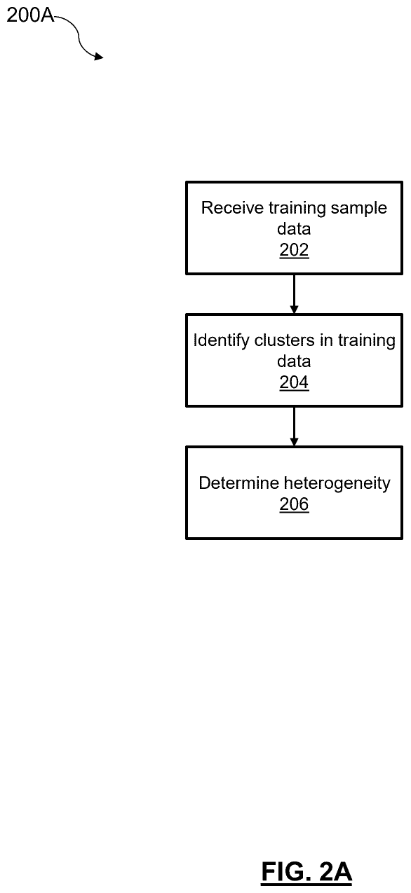

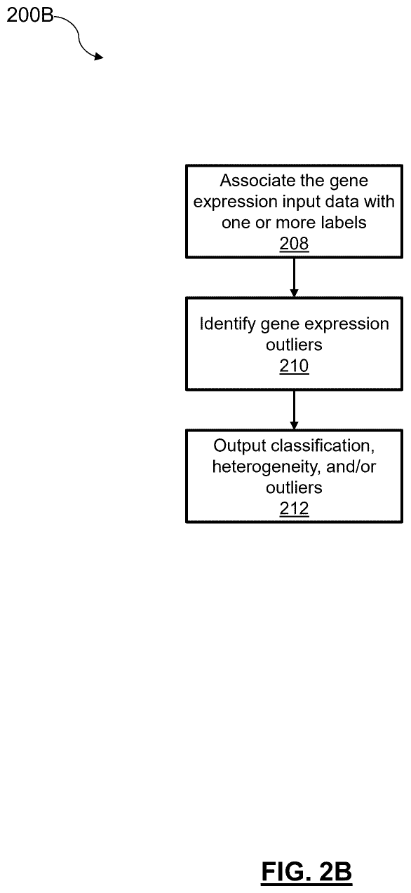

Turning to FIG. 2A, a flowchart 200A for a method for nucleic acid data points based clustering for classifying cancer, according to an embodiment, is shown.

At block 202, the input module 102 receives nucleic acid data points from one or more samples. The samples may be biological samples from one or more subjects. The subjects may be human subjects. The human subjects may be children. In an example, the nucleic acid data points can comprise RNASeq data. The input may be in tabular form with samples (rows) and features (columns). For example, a tabular data set of nucleic acid data points may comprise at least two columns and a plurality of rows, wherein a cell or entry in the tabular data set may be indexed using a row-column (r,c) pair. A first column may comprise a set of features and a second column may comprise a set of values. The features can be genes and the values can be expression counts of such genes. In such example, each sample is a datapoint with normalized gene expression counts as features that can be calculated from, for example, raw RNA-Seq fastq files. In some cases, an RNAseq pipeline can be used that includes STAR (Spliced Transcripts Alignment to a Reference) for the reads alignment and RSEM (RNA-Seq by Expectation Maximization) for the transcriptional counts.

At block 204, the optimization module 104 optimizes the input dataset. The dataset may be optimized by identifying clusters with a density-based approach. In some cases, prior to identifying clusters, the optimization module 104 performs, at least one of, removing low information features and reducing their complexity with a non-linear dimensionality reduction approach. In some cases, identifying clusters can be iterative. At each successive iteration of clustering, a search is performed to identify clusters depth-first for each of the clusters identified at the current level. The search can be terminated when further splits would lead to a particularly suboptimal value of an objective function, and/or the class population is lower than a pre-set bound (e.g. 25-50 samples). The features removal cut-off, the number of neighbours employed by UMAP, and the clusters search parameter (e.g. maximum clustering distance parameter in density-based spatial clustering of applications with noise (DBSCAN)) are identified through an optimization process which tries to maximize a score evaluating the quality of the clustering. In an example, tunable parameters can be optimized with a Grid Search and the total silhouette coefficient of the dataset was set as the objective function. This score quantifies the quality of clustering by calculating the ratio between the clusters' cohesion and their separation. Ranging between −1 when all points are incorrectly assigned, and 1 when all points are well assigned, in an example, 0 can be set as a minimum threshold for accepting a set. In a scenario where the best combinations of parameters found still leads to a negative score, the cluster under scrutiny may not be split.

Removing low information features can use any suitable approach. For example, removing low information features may be performed by ranking the features by their variance and defining a cut-off, and removing features whose variance is below the cut-off. The cut-off can be defined using any suitable approach, such as being a percentage of total variance; defined as the sum of the variance calculated from each feature. After ranking the features and defining the cut-off, their variance is summed in order from the most variant to the least variant. Once a percentage (e.g. 90%) of the total variance is reached, the remaining features are removed. Other information content measures other than variance (e.g. MAD) can be used.

As an alternative to low variance removal, truncated Single Value Decomposition can be used. This is a matrix factorization method similar to PCA that allows decomposing the data into orthogonal features (i.e., have no correlation) and ranking them by variance.

Non-linear dimensionality reduction can use any suitable approach to enhance both efficiency and accuracy. Working in high-dimensions can be computationally expensive and it can lead to overfitting of models, due to the presence of spurious features, and can be difficult to interpret. Furthermore, these datasets are prone to sparsity and distance concentration, which is the convergence of all distances to a similar value (i.e., the curse of dimensionality). In an example, UMAP can be used for dimensionality reduction. The advantage of using non-linear, over linear, approaches is that such approaches allow modelling of high-dimensionality high-complexity datasets and the non-linear relationship among their points with high-fidelity.

In some cases, at each iteration of identifying clusters, the optimization module removes low-information genes, further reduces the dimensionality with the non-linear dimensionality reduction, and applies a clustering search approach to identify the clusters. Each of the above may require some parametrization. In other approaches, parameterization is commonly left to user experience and knowledge of the domain. However, advantageously, in the present embodiments, optimization is performed (e.g., with a grid search) that can be used to search and find the optimal set of parameters. In some cases, the optimization of the parameters can be repeated at each iteration, or at each set of iterations. For each cluster identified at a given iteration, the optimization 11 (e.g., grid search) can be repeated internally, by using only the data included in that cluster. This allows for the identification of structures internal to that group, such as subclusters. The data can then be merged in a hierarchy of clusters. This hierarchy can have levels, containing all of the clusters identified during the first iteration (first level), the second iteration for each cluster identified in the first iteration (second level), the third iteration, and so on. This operation can be carried out depth-first; i.e., once a set of clusters are identified, the optimization module 104 selects one cluster, repeats the operation internally for this cluster, and then further iterates in depth following the branch stemming from that specific cluster. These steps can be performed before moving to the next cluster identified at the first level. In contrast, a breadth-first approach would explore and analyze each cluster at one level before moving to the next level.

The optimization module 104 iterates the clusters search with different parameters, which give raise to different clustering, also referred to as “splits”. In some cases, the quality of the clustering can be evaluated using an internal validation score. The optimization module 104 can use any suitable objective function; in an example, a silhouette score can be used to determine the internal validation score for clustering. This score is calculated for each iteration and the best scoring set of clusters is stored and used. The score ranges from −1 to 1, where 1 corresponds a perfect separation (based on the points distances and arrangement), 0 corresponds to a random arrangement, −1 corresponds to a clustering where all points are matched to the wrong cluster. In a particular case, 0 can be set as a lower boundary, and all clustering selections need to be at least better than a random arrangement, otherwise the parent cluster will not be split. Where no optimal clusters are found, that is when the best choice is not to split a group, the optimization module 104 can stop the search. In some cases, a population cut-off can be used. If a cluster has less then the number of samples corresponding to the cut-off, the optimization module 104 will not attempt to further identify subclusters within such group; i.e., it will not be split.

While a grid search is described herein, it is understood that the optimization module 104 can use any other suitable optimization approach; for example, Differential Evolution, Tree-structured Parzen Estimators, and the like.

### Entropy and Heterogeneity

Expression variance can be used as a proxy of its variability, and it can be easily measured. However, it may fail when dealing with multimodal or discontinuous distributions, and so do its derived quantities (e.g., the coefficient of variation). Shannon's entropy S may be a more appropriate and robust measure. It is a generalization to Boltzmann's thermodynamics entropy, it quantifies the information content, or the randomness of a given distribution. It is defined as follows:

$$S = - {\sum }_{i} {P}_{i} log {P}_{i}$$

where Pᵢ is the probability of an event, i, which is the probability that a certain gene will give a specific expression count.

While entropy entails the overall variability of gene expression within a population, part of this can be translated into a different pattern of activation of significant biological pathways to determine those differences in gene expression that are strong enough to affect biological pathways significantly and to be translated into observable differences in the phenotype; thus, defining different tumor types. This inter-tumoral heterogeneity is explicitly accounted for by the clusters hierarchy itself. Inter-tumoral heterogeneity is, in this context, a set of transcriptional differences that are strong and coherent enough to allow for the identification of different groups of samples with common characteristics. This information can be used to define and separate these groups and build the hierarchy. In contrast, intra-tumoral heterogeneity refers to those expression differences across samples in the same tumour type that, however, cannot be leveraged to define subtypes and further split the cluster.

In some cases, at block 206, the heterogeneity module 106 can define a score based on the number of offspring nodes that a specific group generates and measure it on the classes identified. This score can be used to summarize the number of offspring subclusters stemming from a given cluster in the hierarchy. This number can be weighted by the population of each cluster because it can be expected that larger clusters will separate more easily than smaller clusters. The score can thus be used to summarize the complexity of a hierarchical branch coming from a given cluster. Groups with a high score can have a lot of subtypes and sub-subtypes and have a complex hierarchy below. Lower scores indicate few to no subtypes. This score can be informally referred to as Population Weighted Splits (PaWS) and defined as follows:

$$PaWS ( n ) = \frac{\vert {L}_{n} \vert}{\vert L \vert} \frac{log ( \vert root \vert )}{log ( \vert n \vert )}$$

where n={sample₁, sample₂, . . . } is a set of samples identified as a cluster or node, L is the set of all leaf nodes l, that is all the childless nodes, Lₙ is the set of leaf nodes that are offspring of n, and root is the hierarchical tree root, a set containing all the dataset samples. The PaWS of n is thus defined as the ratio between the cardinality (|Lₙ|) of Lₙ, that is the number of leaf nodes that are offspring of n, over the total number of leaf nodes, weighted by the inverse of the log population ratio of n. This last term is added in this case to account for the fact that smaller clusters will have less probability to be split.

The correlation between heterogeneity scores provides a substantial benefit. In the example experiments, it was observed that there was a weak correlation (Spearman's rank test coefficient=0.355, p-val=1.120e-05) between median entropy and PaWS score; after removing all the leaf nodes to avoid including clusters with possible subtypes but insufficient population to be split. Entropy proved thus to be a good proxy for intra-cluster expression disorder, as it accounts for that part of expression differences within a population that are not coherent enough to be translated into clear subtypes and yet not able to disrupt the overarching patterns that define the parent class.

Turning to the flow diagram 200B of FIG. 2B, at block 208, the classification module 108 uses a machine learning model, for example an ensemble of convolutional neural networks (CNN), to classify gene expression input data as corresponding to one or more labels corresponding with the clusters determined from the flow diagram 200A of FIG. 2A. The labels may be associated with types or subtypes of cancerous tumors. In most cases, the ensemble classifier takes an expression vector as input; i.e., for each sample, a vector containing TPM (transcripts per million) normalized expression counts for each gene. Advantageously, the classification module 108 does not blindly rely on given labels for the data in a fully supervised fashion. Such labels may be hard to come by and often come with inaccuracies and a lack of details. The training data of the CNN classifier can be the reference cohort of pan-cancer gene expression data (e.g., the gene expression data used to generate the clusters informing the labels). The target labels are, in this case, the identified classes that make up the hierarchy.

Advantageously, the classification module 108 can use a relatively large expression of protein coding genes and pseudogenes in input (for example, more than 18,000), while other approaches generally rely on a relatively small set of preselected genes. Often a model optimized for a completely different task (such as image classification) is hurriedly readapted for gene expression data, hampering its accuracy. The classification module 108 takes particular care to address the population imbalance that stems from such a complex hierarchy of subclasses (classes at the first levels are invariably more populous and easier to identify than specific and considerably smaller subtypes at the deeper levels). The use of an ensemble of three models rather than a single model allows the classification module 108 to boost accuracy and robustness of the classification. The models may be multilabel, allowing for the identification of multiple tumor components when present. Furthermore, in some cases, the inclusion of healthy normal tissue in a training cohort allows the classifier to recognize normal contamination in tumor samples. In some cases, to account for various nuances of a complex neoplastic environment, the output can be a probability, rather than forcing a binary classification. In some classes, the models may be multiclass, predicting a probability of one tumor being present out of a choice of two or greater classes of tumor.

In an example, the classification module 108 can use a set of mono-dimensional convolutional neural networks (1D-CNN) that takes gene expression reduced output (for example, RSEM (RNA-Seq by Expectation-Maximization) 18,010 log 2-TPM genes) as input and returns the membership probability to any or multiple of the 455 identified classes. In an example, training of the neural network can use a reference pan-cancer gene expression dataset, which can be built by merging data from various sources. Which can be the same dataset that was used to build the clustering hierarchy. The target can be the hierarchy of classes identified herein. The input format can be TPM-normalized expression tables; the same used for the clustering analysis.

In an example, the model can be an ensemble of three CNNs. Each CNN can be composed of 1 to 3 modules, each composed by a one-dimensional (1D) convolutional layer, followed by batch normalization and max pooling. In some cases, an extra convolutional layer, for the identification of large-scale patterns, can be added at the beginning of the model. These layers can be followed by 1 or 2 fully connected layers with dropout, whose size is reduced by, for example, 30% for each subsequent layer. The final layer can also be a fully connected layer.

In other cases, any suitable machine learning architecture can be used; for example, fully connected networks. CNNs may identify correlation patterns among genes at different scales and they require a lower number of parameters to train and are thus more efficient.

In some cases, the models used by the classification module 108 can be built as multilabel, meaning that both the input labels and the output membership assignment are not exclusive to a single class; which is beneficial due to the hierarchical structure of the target. In some cases, post-processing can be performed to ensure consistency among the probabilities of classes within a family. For such post-processing, where the classifier assigned higher probability to an offspring node than to its parent, the average of the two is assigned to both classes and the sibling nodes are adjusted accordingly. Starting from the leaf nodes, this correction is propagated upward along the hierarchy.

In the above cases, multilabel can be understood to mean that each sample used can be assigned to more than one cluster when passed through the classifier. This can help identify the presence of multiple components; for example, if a sample has normal contamination, the classifier will be able to detect both the presence of normal tissue and the tumour. In the above cases, multiclass can be understood to mean that the input data used in the training may not have necessarily had an assignment to a single target class, but multiple classes. This is due to the hierarchical nature of the class assignment. A sample belongs to a class but also to its family (parent class, grandparent class, and so on) along the branch that is followed for a specific tumour type. In this context, the classes can be defined as the clusters membership for the hierarchy of clusters identified by the system.

Adjusting the sibling nodes includes, where a sample is assigned to a parent class and then to more than one of its children, adjusting the probabilities so that the maximum probability of any child is not greater than the probability of the parent. For example, it would not be useful to have a sample be assigned a 100% probability to myeloid leukemia, but then only a 30% probability to leukemia. The system is able to spot this rare occurrence, since the classifier is able to recognize samples belonging to both parent and children classes correctly, and has been observed only in exceptional cases.

In some cases, the probabilities in output from the classifier may not be binarized. This allows the user to evaluate the confidence that each tumor or normal population may be present in the sample without automatically discarding lower probability matches. However, in some cases, to make up for strong class imbalances in the training dataset, and the resulting variability in performance scores across the hierarchy of target classes, these output probabilities can be recalibrated. To recalibrate, a binary classification cut-off is identified that maximizes an adapted Youden J statistic (precision+recall) and then the output scores are adapted linearly so that this cut-off value falls at, for example, 0.5 of the final probability. While this change does not drastically affect the resulting output (as observed on the validation cohort of the example experiments, 0.998 cosine similarity, 0.957 hierarchical similarity), it helps relieve some of the overfitting on minority classes. The median cut-off in the example experiments was 0.585 (0.651 for highest level classes, 0.425 for the lowest level, 0.543 for leaf nodes) suggesting this is an overall balanced classifier. In some cases, the input data features (for example, genes) for the classifier can

be ordered by the classification module 108 by correlation following an agglomerative clustering on their log 2 normalized expressions. The input data features can be ordered by measuring their pairwise expression correlation across the training dataset and running agglomerative clustering. Genes that are more correlated will be positioned closed together. In this case, the input gene arrays are scaled to a zero to one range and the labels are transformed to a one-hot boolean encoding.

For the present embodiments, where the prediction can be carried out on a large number of overlapping classes, the hierarchical similarity score can be an approximation that ranges between 0 and 1, with 1 representing maximal similarity.

At block 210, in some cases, the outlier module 110 uses a framework that uses the information coming from the classification module 108 to identify gene expression outliers in the input sample data. In this way, the classification can be used to build a matching tumour type distribution for a comparison of the gene expression of specific genes in the sample. The outlier detection can be used to identify those genes in the distribution that are aberrantly expressed with statistical significance. The outlier module 110 compares gene expression observed in a sample under investigation against gene expression distributions of reference tumours and normal tissues, and p-values are extracted. The gene expression distributions can be obtained by building mixture datasets starting from a reference cohort. The ratios of these mixtures can be based on the identified probabilities from the classification module 108. In an example, the healthy normal tissues comparison, in particular, can exploit forced matching to a list of normals classes by means of a kNN classifier.

In a particular case, the framework used by the outlier module 110 can compare the expression of a gene of interest, within a sample, against reference distributions based on the assignment probabilities obtained by the gene expression classifier. These distributions can include matching of the tumour types identified by the classifier, but also matching with the remaining tumour types in the reference dataset and the closest matching normal tissue. These distributions can be built as a mixture of samples from the reference dataset belonging to these different groups. Their population ratio can correspond to the probabilities of assignment to different classes in output from the classifier. For example, if a sample is assigned with 80% confidence to leukemia and 20% confidence to sarcoma, the distribution will contain 80% samples form leukemias and 20% samples from sarcomas. The closest matching normal group match can be obtained, for example, with a k-NN classifier trained only on the healthy normal tissue samples subset and the normal tissue branch of the hierarchy. The distributions can be approximated with a kernel density algorithm to assist in cases where the samples number are low. The expression of the sample can then be directly compared and p-values can be obtained with, for example, a Monte Carlo approach.

As an example, if a sample is classified by the classification module 108 as containing 70% leukemia and 30% sarcoma, the expression of chosen genes can be compared against the following mixture populations:

The closest normal matching, most probably whole blood given its majority tumour class identified as leukemia. 16
A random subset of leukemia samples and subsets of all its identified subtypes separately.
A random subset of sarcoma samples and subsets of all its identified subtypes separately.
A mixture of leukemias and sarcomas with a 70-30 proportion.

Generally, populations show different facets of the expression analysis. Comparing against matching normals helps to better understand if the gene is overexpressed in the tumour exclusively or if it is also commonly expressed in a healthy individual. Comparing against matching tumours helps to better understand if the gene is an outlier, even within the putative tumour type of the sample. This can suggest the presence of aberrant expression that is not typical in the identified tumour subtype but its idiosyncratic of the sample. Comparing against non-matching tumours helps to better identify if the gene is commonly expressed in other tumour types. In some cases, the datasets can be sub-selected randomly to improve computation times.

These multiple comparisons, together with the information coming from the classification module 108, provide a complete assessment of the sample's transcriptional behaviour. A gene could be overexpressed significantly against certain tumour types identified, but not against others. The above approach can be repeated for a gene sets enrichment analysis (GSEA) for identification of differentially expressed pathways using statistical approaches to identify significantly enriched or depleted groups of genes.

At block 212, the output module 112 outputs one or more of the classifications from the classification module 108, the identified outliers from the outlier module 110, the accuracy of predictions from the classification module 108, and the heterogeneity scores from the heterogeneity module 106, to the interface module 168, the network module 176, and/or the database 188.

While the present embodiments are generally directed to the classification of cancers for the purposes of diagnosis, it is understood that, for certain subtypes of cancer, classification can further include determining prognostic and/or therapeutic information based on the diagnosis. For example, diagnosis of a certain subtype May have an associated reduced survival rate and/or be vulnerable to a given drug.

### Benign and Malignant Neoplasm

In further embodiments, other machine learning approaches can be used. In a particular case, Gradient Boosting (GB) machine learning approaches can be used in an example application in order to differentiate between benign and malignant thyroid neoplasm.

With respect to such example application, the recorded incidence of thyroid carcinomas in, for example, the North American population has been increasing in the past few decades across all age groups, and so the efforts to develop quick, accurate, and possibly minimally-invasive tests to evaluate the malignancy of thyroid nodules has become imperative. Up to 65% of the adult population is now expected to carry nodules in the thyroid gland. Ultrasound and fine-needle aspiration (FNA) biopsies are part of common routine tests to identify their risk group, but yield an indeterminate results in up to a quarter of the cases. Only an estimate of one fifth of the patients undergoing thyroidectomy harbour a true malignancy, while most still undergo an unnecessary surgery.

Various classifiers, based on microarray expression of limited panels of marker genes, have been attempted to find preoperative tests of the severity of thyroid nodules. The Afirma™ Gene Expression Classifier (AGEC), in particular, can be used for separation of benign and ‘suspicious’ nodules. Various reports confirm the impact and clinical utility of the AGEC test as having a 92% sensitivity, a 52% specificity in recognizing suspicious nodules, and a 95% negative predictive value (NPV).

Applying the scale-adaptive hierarchical clustering framework, described herein, with the Gradient Boosting (GB) machine learning approaches, provided below, transcriptional classification of thyroid cancer can be determined. The scale-adaptive hierarchical clustering framework can be used to build a comprehensive hierarchy of thyroid carcinoma subtypes, such that the system is able to characterize idiosyncratic expression profiles, to correlate them to known and novel marker mutations and clinical features, and to detail their relative distance within the transcriptional space. In the present application, limiting the scope to a specific tumour type can be used to focus on detailing the hierarchical relationship among the tumour subtypes. In the present application, to amplify the reach of this cluster analysis, example experiments used an extensive cohort of 860 samples, including papillary thyroid carcinoma (PTC), follicular variant PTC (FVPTC), follicular carcinoma (FTC), follicular adenoma (FA), healthy normal tissue (HN) and tumour-adjacent normal tissue samples (TAN) from a variety of sources.

In a particular case, the system can train a set of three Gradient Boosting (GB) classifiers on a cohort: a first binary model to separate benign from malignant nodules, a second model to separate healthy normal from tumour-adjacent normal tissues, and a third multiclass model targeting first layers of a thyroid carcinoma transcriptional hierarchy. Importance scores can be extracted from the trained model to identify features (i.e., genes) that provide the greatest contribution to the definition of each group.

Generally, Gradient Boosting robust predictions come from an ensemble of weak learners, such as decision trees, and applied to classification and regression tasks. In the example experiments, GB leads to an improvement in specificity while maintaining an effective NPV. The present embodiments were determined to provide a fast and inexpensive complement to other classification approaches and demonstrates substantial value of RNA-Seq whole transcriptome expression data when compared to limited panels of manually curated genes. The present embodiments advantageously provide high performing approaches for the preoperative identification of nodules malignancy.

In some cases, the optimization module 104 can use multi-level clustering independently to the normal tissues samples and malignant subsets. Normal and tumor samples from the same organ would have been grouped in common classes at the highest level if they had been mixed. Clustering quality decreases as less aggressive, or low-purity tumours can be difficult to separate from healthy normal samples. During training, this design decision can advantageously force the classification module 108 to learn high-level features that distinguish normal tissue from neoplasms, independently from their anatomical location. In this way, the classification module 108 can better recognize tumor populations in low-quality samples, as the tumor-normal separation is prioritized in the hierarchy. Similarly, generalization to unseen normal tissues will also generally be improved.

In many cases, clusters obtained by the optimization module 104 can be annotated based on their most characteristic transcriptional features compared to the closest members of their hierarchical family. Differential gene expression and GSEA can be carried out to identify each cluster's defining gene and pathway expression. Limited clinical information, including age, sex, and a diagnostic label, may be available for each sample. Such annotation can be used to assign a unique label (a code and a name) and the annotation can be extended for families of tumours (for example, for the five major families of pediatric tumors: CNS; leukemia; neuroblastoma; and the two mesodermal classes, as well as the branch stemming from the healthy normal samples).

### Method for Classifying Benign or Malignant Neoplasm

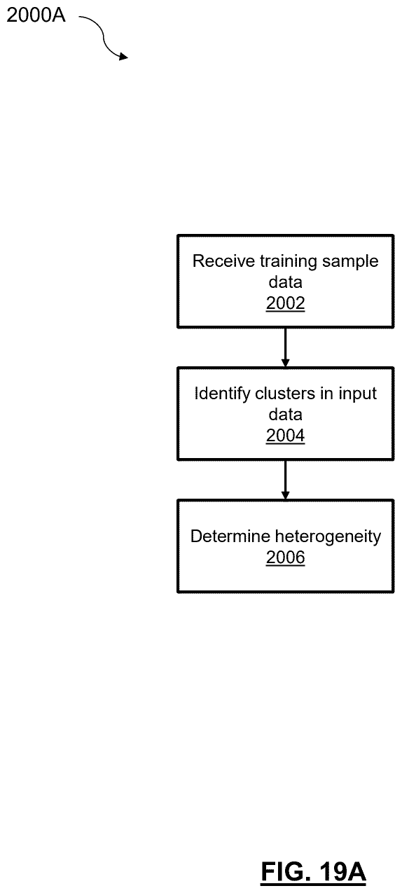

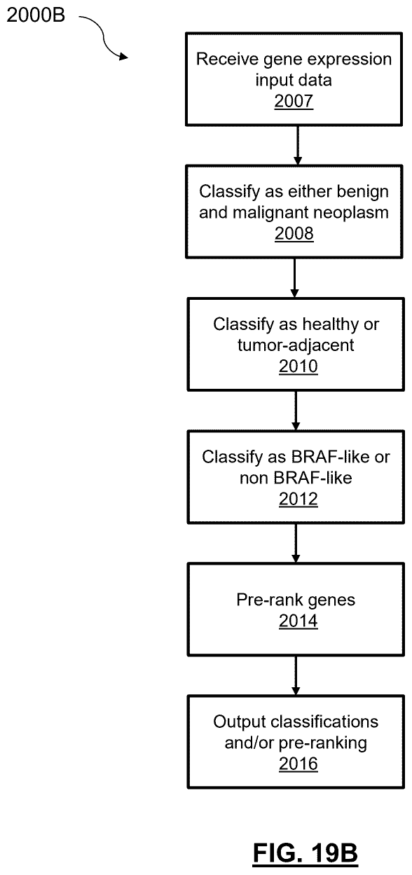

FIG. 19A is a flowchart illustrating a method 2000A for identifying clusters for classifying benign and malignant neoplasm. At block 2002, the input module 102 receives training data comprising nucleic acid data points from one or more samples comprising a whole, or a substantial portion of (e.g., greater than 90%), of a transcriptome. At block 2004, the optimization module 104 optimizes the input dataset by identifying clusters with a density-based approach, as described herein. At block 2006, the heterogeneity module 106 defines a score based on the number of offspring nodes that a specific group generates and measures it on the classes identified; as described herein.

FIG. 19B is a flowchart illustrating a method 2000B for classifying benign and malignant neoplasm, using the method 2000A. At block 2007, the input module 102 receives input data comprising gene expression input data. At block 2008, the classification module 108 uses a first machine learning model, such as using a Gradient Boosting ensemble, to classify gene expression input data as either benign or malignant neoplasm. The classification module 108 builds a set of classifiers and uses their learned weights to confirm the expression pathways most relevant to separate the two groups (benign and malignant). The use of Gradient Boosting (referred to as ‘GBoost’) provides simplification and allows for the interpretation of the results. GBoost is a tree-based machine learning algorithm for classification and regression that is generally more robust than other neural networks on a limited training samples population. Using GBoost, the classification module 108 can efficiently extract feature importance scores in a clear and human-friendly format; which is generally not possible for more complex convolutional networks.

At block 2010, in some cases, the classification module 108 uses a second machine learning model, comprising a Gradient Boosting ensemble, to classify the clustered data as either healthy or tumour-adjacent normal tissues.

At block 2012, in some cases, the classification module 108 uses a third machine learning model, comprising a Gradient Boosting multiclass model ensemble, to classify the gene expression input data to separate all of these four classes (benign, malignant, healthy, and tumour-adjacent normal tissue) and split tumours in BRAF-like and non BRAF-like.

At block 2014, in some cases, a gene sets enrichment analysis can be performed by the classification module 108 to pre-rank genes based on their importance in each of these models.

At block 2016, the output module 112 outputs the classification of benign or malignant, and in some cases, also outputs one or more of the healthy or tumour-adjacent normal tissues classifications, the BRAF-like and non BRAF-like classifications, and the pre-ranking of genes.

### Particular Implementations

In an aspect, there is provided a computer-implemented method for classification of cancer from gene expression input data, the method comprising: receiving a training dataset comprising nucleic acid data points from one or more samples; identifying classes in the training dataset comprising performing recursive clustering by, at each successive iteration, performing a search to identify clusters based on similarity, wherein each class of the classes is associated with a tumor; training a machine learning model to associate the nucleic acid data points of the training dataset with the identified classes; receiving the gene expression input data for classification; classifying, using the trained machine learning model, the gene expression input data as one or more of the identified classes; and outputting the classification of the gene expression input data. In a particular case of the method, the method further comprises, prior to identifying classes, performing removal of low information features. In another case of the method, removal of low information features comprises ranking features by their respective variance and removing features whose variance is below a pre-determined cut-off. In yet another case of the method, the variance is determined using Shannon's entropy. In yet another case of the method, the method further comprises, prior to identifying the classes, performing non-linear dimensionality reduction. In yet another case of the method, non-linear dimensionality reduction comprises performing Uniform Manifold Approximation and Projection. In yet another case of the method, optimizing the input dataset comprises determining a score representative of a quality of the identified clusters by determining a ratio between cohesion of the clusters and separation of the clusters. In yet another case of the method, the method further comprises performing a grid search to determine optimized parameters for identifying the clusters. In yet another case of the method, determining the optimized parameters is repeated for each iteration using only data included in a given cluster to identify hierarchical subclusters. In yet another case of the method, once clusters and subclusters are identified, each cluster is successively selected and parameters are optimized internally for such cluster, further iterations of optimization are performed following a branch stemming from such cluster. In yet another case of the method, performing recursive clustering comprises: iteratively identifying clusters with different parameters for each iteration; evaluating each identification of clusters using an internal validation score; and selecting the identification of clusters with the highest score. In yet another case of the method, the method further comprises determining complexity of a hierarchical branch stemming from each cluster based on a number of offspring hierarchical nodes. In yet another case of the method, the trained machine learning model outputs membership probability to one or more of the identified classes. In yet another case of the method, the trained machine learning model comprises an ensemble of convolutional neural networks. In yet another case of the method, each convolutional neural network comprises one or more one-dimensional convolutional layers, followed by one or more fully connected layers with dropout, and followed by a fully connected layer. In yet another case of the method, the trained machine learning model is multiclass such that the gene expression input data can be assigned to more than one class. In yet another case of the method, the method further comprises performing agglomerative clustering on log 2 normalized expressions of the gene expression input data prior to classification. In yet another case of the method, the input dataset comprises genes as features and expression counts of such genes as values.

In yet another case of the method, the reference dataset comprises gene expression from healthy tissue. In yet another case of the method, the method further comprises identifying gene expression outliers in the gene expression input data prior to classification, comprising comparing gene expression for the input data against gene expression distributions of reference tumours and normal tissues. In yet another case of the method, each of the classes are associated with a type of sarcoma tumor. In yet another case of the method, the classes are associated with one or more of osteosarcoma, leiomyosarcoma, fusion-positive rhabdomyosarcoma, fusion-negative rhabdomyosarcoma, synovial sarcoma, and Ewing sarcoma. In another aspect, there is provided a system for classification of cancer from gene expression input data, the 8 system comprising: an input module to receive a training dataset comprising nucleic acid data points from one or more samples and receive the gene expression input data for classification; an optimization module to identify classes in the training dataset comprising performing recursive clustering by, at each successive iteration, performing a search to identify clusters based on similarity, wherein each class of the classes is associated with a tumor; a training module to train a machine learning algorithm to associate the nucleic acid datapoints of the training dataset with the identified classes; a classification module to classify, using the trained machine learning model, the gene expression input data as one or more of the identified classes; and an output module to output the classification of the gene expression input data. In a particular case of the system, the optimization module, prior to identifying classes, performs removal of low information features. In another case of the system, the removal of low information features comprises ranking features by their respective variance and removing features whose variance is below a pre-determined cut-off. In yet another case of the system, the variance is determined using Shannon's entropy. In yet another case of the system, the optimization module, prior to identifying classes, performs non-linear dimensionality reduction. In yet another case of the system, the non-linear dimensionality reduction comprises performing Uniform Manifold Approximation and Projection. In yet another case of the system, optimizing the input dataset comprises determining a score representative of a quality of the identified clusters by determining a ratio between cohesion of the clusters and separation of the clusters. In yet another case of the system, the optimization module performs a grid search to determine optimized parameters for identifying the clusters. In yet another case of the system, determining the optimized parameters is repeated for each iteration using only data included in a given cluster to identify hierarchical subclusters. In yet another case of the system, once clusters and subclusters are identified, each cluster is successively selected and parameters are optimized internally for such cluster, further iterations of optimization are performed following a branch stemming from such cluster. In yet another case of the system, performing recursive clustering comprises: iteratively identifying clusters with different parameters for each iteration; evaluating each identification of clusters using an internal validation score; and selecting the identification of clusters with the highest score. In yet another case of the system, the optimization module further determines complexity of a hierarchical branch stemming from each cluster based on a number of offspring hierarchical nodes. In yet another case of the system, the trained machine learning model outputs membership probability to one or more of the identified classes. In yet another case of the system, the trained machine learning model comprises an ensemble of convolutional neural networks. In yet another case of the system, each convolutional neural network comprises one or more one-dimensional convolutional layers, followed by one or more fully connected layers with dropout, and followed by a fully connected layer. In yet another case of the system, the trained machine learning model is multiclass such that the gene expression input data can be assigned to more than one class. In yet another case of the system, the classification module further performs agglomerative clustering on log 2 normalized expressions of the gene expression input data prior to classification. In yet another case of the system, the reference dataset comprises gene expression from healthy tissue. In yet another case of the system, the system further comprises an outlier module to identify gene expression outliers in the gene expression input data prior to classification, comprising comparing gene expression for the input data against gene expression distributions of reference tumours and normal tissues. In yet another case of the system, each of the 26 classes are associated with a type of sarcoma tumor. In yet another case of the system, the classes are associated with one or more of osteosarcoma, leiomyosarcoma, fusion-positive rhabdomyosarcoma, fusion-negative rhabdomyosarcoma, synovial sarcoma, and Ewing sarcoma. In yet another aspect, there is provided a computer-implemented method for classifying benign and malignant neoplasm from gene expression input data, the method comprising: receiving a training dataset comprising nucleic acid data points comprising a whole, or a substantial portion of, a transcriptome; identifying classes in the training dataset comprising performing recursive clustering by, at each successive iteration, performing a search to identify clusters based on similarity; training a machine learning model to associate the nucleic acid data points of the training dataset with the identified classes; classifying the gene expression input data as either benign or malignant neoplasm using the trained machine learning model; outputting the classifications of benign or malignant. In a particular case of the method, the machine learning model comprises a Gradient Boosting ensemble, the Gradient Boosting ensemble comprises a set of classifiers and the learned weights of the classifiers are used to confirm expression pathways relevant to benign and malignant classifications. In another case of the method, outputting the classifications of benign or malignant comprises outputting feature importance scores extracted from the classifications of the clusters. In yet another case of the method, classifying the clusters as either benign or malignant neoplasm using the trained machine learning model further comprises extracting immune activity, interleukin and integrin signaling, and markers of thyroid carcinoma based on the classification. In yet another case of the method, the method further comprises classifying the clustered data as either healthy or tumour-adjacent normal tissues using a second machine learning model, the second machine learning model comprising a Gradient Boosting ensemble, and outputting the classification as either healthy or tumour-adjacent normal tissues. In yet another case of the method, classifying the clustered data as either healthy or tumour-adjacent normal tissues using the second machine learning model further comprises extracting RAS and ESR1 pathways, cell adhesion, and locomotion and metabolic pathways based on the classification. In yet another case of the method, the method further comprises classifying the classified benign or malignant neoplasm as either BRAF-like and non-BRAF-like using a third machine learning model, the third machine learning model comprising a Gradient Boosting ensemble, and outputting the classification as either BRAF-like and non-BRAF-like. In yet another case of the method, classifying the classified benign or malignant neoplasm as either BRAF-like and non-BRAF-like using the third machine learning model further comprises extracting mammalian target of rapamycin (mTOR), GTPase and cell cycle based on the classification. In yet another case of the method, the input dataset comprises nucleic acid data from healthy samples, and wherein the healthy samples are also classified as either BRAF-like and non-BRAF-like. In yet another case of the method, the input dataset comprises nucleic acid data from thyroid samples. In yet another aspect, there is provided a system for classification of benign and malignant neoplasm from gene expression input data, the system comprising one or more processors in communication with data storage to execute: an input module to receive a training dataset comprising nucleic acid data 8 points comprising a whole, or a substantial portion of, a transcriptome; an optimization module to identify classes in the training dataset comprising performing recursive clustering by, at each successive iteration, performing a search to identify clusters based on similarity; a training module to train a machine learning model comprising a Gradient Boosting ensemble to associate the nucleic acid data points of the training dataset with the identified classes; a classification module to classify the gene expression input data as either benign or malignant neoplasm using the trained machine learning model; and an output module to output the classifications of benign or malignant. In a particular case of the system, the machine learning model comprising a Gradient Boosting ensemble, the Gradient Boosting ensemble comprises a set of classifiers and the learned weights of the classifiers are used to confirm expression pathways relevant to benign and malignant classifications. In another case of the system, outputting the classifications of benign or malignant comprises outputting feature importance scores extracted from the classifications of the clusters. In yet another case of the system, classifying the clusters as either benign or malignant neoplasm using the trained machine learning model further comprises extracting immune activity, interleukin and integrin signaling, and markers of thyroid carcinoma based on the classification. In yet another case of the system, the classification module further classifies the clustered data as either healthy or tumour-adjacent normal tissues using a second machine learning model, the second machine learning model comprising a Gradient Boosting ensemble, and outputting the classification as either healthy or tumour-adjacent normal tissues. In yet another case of the system, classifying the clustered data as either healthy or tumour-adjacent normal tissues using the second machine learning model further comprises extracting RAS and ESR1 pathways, cell adhesion, and locomotion and metabolic pathways based on the classification. In yet another case of the system, the classification module further classifies the classified benign or malignant neoplasm as either BRAF-like and non-BRAF-like using a third machine learning model, the third machine learning model comprising a Gradient Boosting ensemble, and outputting the classification as either BRAF-like and non-BRAF-like. In yet another case of the system, classifying the classified benign or malignant neoplasm as either BRAF-like and non-BRAF-like using the third machine learning model further comprises extracting mammalian target of rapamycin (mTOR), GTPase and cell cycle based on the classification. In yet another case of the system, the input dataset comprises nucleic acid data from healthy samples, and wherein the healthy samples are also classified as either BRAF-like and non-BRAF-like. In yet another case of the system, the input dataset comprises nucleic acid data from thyroid samples. In yet another aspect, there is provided a system for classification of a tumor from gene expression input data, comprising: (a) a clustering module; and (b) a classification module; wherein the clustering module performs one or more operations comprising generating a hierarchy of labels, wherein a label of the hierarchy of labels is associated with a tumor; wherein the classification module performs one or more operations comprising: training a classification model to associate gene expression training data with one or more labels of the hierarchy of labels; and associating the gene expression input data with one or more labels of the hierarchy of labels by processing the gene expression input data with the trained classification model. In a particular case of the system, the gene expression input data comprises RNA sequence data. In another case of the system, the RNA sequence data comprises one or more of polypeptide encoding sequence data and pseudogene sequence data. In yet another case of the system, the RNA sequence data comprises sequence data encoding or corresponding with more than 18,000 polypeptides and/or pseudogenes. In yet another case of the system, polypeptide encoding sequence data comprises sequence data encoding a plurality of genes and corresponding gene expression counts. In yet another case of the system, generating the ground truth data comprises using agglomerative clustering on log 2 normalized expressions of the gene expression input data prior to classification. In yet another case of the system, generating the ground truth data comprises iteratively clustering the gene expression input data, wherein during an iteration a score is calculated for a set of clusters. In yet another case of the system, generating the ground truth data further comprises selecting a set of clusters with a highest score. In yet another case of the system, the score is based at least in part on a ratio between cohesion of the clusters and separation of the clusters. In yet another case of the system, iteratively clustering the gene expression data is performed using a grid search or tree-based method. In yet another case of the system, the hierarchy 8 comprises at least a first level and a second level. In yet another case of the system, a label corresponding to a first class of tumor is associated with the first level and a label corresponding to a second class of tumor is associated with the second level, wherein the first class comprises the second class. In yet another case of the system, the first class of tumor corresponds to a cluster of the gene expression input data, wherein the cluster groups a plurality of gene expression data items based at least in part on their similarity. In yet another case of the system, the second class of tumor corresponds to a subset of the cluster, wherein the subcluster groups a plurality of gene expression data items of the cluster based at least in part on their similarity. In yet another case of the system, the classification model is a multiclass model. In yet another case of the system, the classification model is an ensemble model. In yet another case of the system, the ensemble model comprises a plurality of neural networks. In yet another case of the system, the neural networks are convolutional neural networks (CNNs). In yet another case of the system, a convolutional neural network comprises one or more one-dimensional (1D) convolutional layers, one or more fully-connected layers with dropout, and a fully-connected layer. In yet another case of the system, generating the ground truth data comprises removing low information features from the gene expression input data. In yet another case of the system, removing low information features comprises (1) defining a threshold variance and (2) removing features of the gene expression input data with variances lower than the threshold variance. In yet another case of the system, the variances are determined based at least in part using Shannon's entropy. In yet another case of the system, generating the ground truth data comprises applying non-linear dimensionality reduction to the gene expression input data. In yet another case of the system, the non-linear dimensionality reduction comprises Uniform Manifold Approximation and Projection (UMAP). In yet another case of the system, matching the tumor with the one or more labels comprises generating a set of probabilities, a probability of the set of the probabilities indicating a quantitative degree of association between the tumor and at least one label of the one or more labels. In yet another case of the system, the hierarchy of labels does not include any manual or human-generated labels. In yet another aspect, there is provided a method for classification of a tumor from gene expression input data, performed using a system comprising a clustering module and a classification module, comprising: generating, by the clustering module, a hierarchy of labels, wherein a label of the hierarchy of labels is associated with a tumor; training, by the classification module, a classification model to associate gene expression training data with one or more labels of the hierarchy of labels; and associating, by the classification module, the gene expression input data with one or more labels of the hierarchy of labels by processing the gene expression input data with the trained classification model. In another case of the method, the method further comprises determining or modifying a course of treatment based at least in part on the association of the gene expression input data with the one or more labels.

### Experiments

The following paragraphs describe experiments and experimental results and should not be construed as to limit any aspects of the preceding disclosure.

### Summary of Experiments for Classifying Childhood Cancer

Example experiments determined that the disclosed system can yield a hierarchy of 455 tumor and normal classes when applied to a comprehensive cohort of 13,313 samples; where the more samples that are RNA sequenced, the more classes that can be created. When applied to a hard-to-treat cohort in the example experiments, the OTTER classifier was concordant with clinical pathology in 78% of ongoing patients and helped clarify the diagnosis of 7% of the cases. The classifier was blinded to the diagnosis provided by the pathologist.

The example experiments performed the clustering process described herein on a reference set of 2,178 childhood and 9,400 adult tumors and 1,735 non-neoplastic samples revealed a hierarchy of 455 clusters (or classes), representing 406 types of human cancer and 49 classes of normal tissue (discussed in detail below). The average silhouette coefficient, a score quantifying how distinct clusters are one from another, was determined to be high across all tumor types and subtypes, thus providing an internal validation for these clusters.

### Clustering

The example experiments determined the OTTER CNN ensemble classifier to achieve higher scores across all evaluation metrics than any single model did on its own (0.955 5×5-fold validation median MAUCPR and 0.997 MF1 after calibration).

The example experiments also investigated the behaviour of the classifier of the present embodiments in inference on data from tumour types missing from the transcriptional phenotypes' hierarchy, and the effect of adding such data to the clustering was determined. To this end, 19 atypical teratoid/rhabdoid tumors (AT/RT) from an unseen dataset was selected, which was not used for clustering or training, and were run through the classifier. 18 AT/RTs snap frozen tumor materials and clinical information were collected and all AT/RTs had negative BAF47 immunohistochemistry stain and biallelic SMARCB1 inactivating alterations as confirmed with FISH, MLPA, targeted Sanger sequencing, or high-throughput sequencing analyses. The reference cohort contained 3 samples that had been labelled as AT/RT. Two of these were found in T040 GLI HG/GBM MES, a group of high-grade gliomas and glioblastomas of the mesenchymal subtypes. The remaining samples were grouped with lung adenocarcinoma, likely due to contamination or low tumour purity. 15 out of 19 samples were assigned to classes along the T040 CNS branch with at least 5% of confidence. AT/RTs also possessed some signal of high stemness, yielding a partial match to the mesodermal stem high class (T004) in 15/19 samples. The group of 21 AT/RTs (19 unseen+2 high quality samples from the reference cohort) were clustered together with samples from the CNS class.

All AT/RTs were clustered together within a new class (just below the high-grade gliomas T034; in the same lineage as T040). This demonstrates that a critical threshold of ATRTs was reached to create a new subtype. To study what the exact threshold was, subsetting experiments were performed. Clustering was repeated using different numbers of AT/RTs samples along with 100 other CNS tumors, both of which had been randomly selected five separate times. Tree-Parzen Estimators were used to speed up the search for repeated runs. The Adjusted Mutual Information score (AMI) on the clustering result was determined by assuming a perfect separation of AT/RTs from all other CNS samples as ground truth, in order to assess how close the resulting partition was to having an AT/RTs-only class.

In the example experiments, the optimization module 104 was applied on the input dataset to build a hierarchical tree of tumor and normal subtype clusters. In this example, the number of final (reduced) dimensions was empirically set to 12 as a compromise between accuracy and computational cost; but in other cases, and suitable cut-off can be used. A population cut-off of 25 was applied to stop the search and nodes with less than 10 samples were pruned, since their annotation and training for the classifier would be too unreliable. In this example, these parameters yielded more than individual clusters. In some cases, a subset of low-population leaf nodes can be removed for the lack of sufficient biological and gene expression information, together with classes populated exclusively by ribodepleted samples. The cut-off for leaf pruning can be chosen empirically, for example, based on the ability to annotate and characterize the clusters with differential expression (DE) analysis. In some cases, a stricter cut-off can be used to focus on classes with more robust numbers. The ribodepleted samples can be removed, in some cases, due to issues with their quality and batch effects. The present inventors observed that ribodepleted samples and poly(A) samples were separating even when carrying the same disease type. The ribodepleted samples were fewer in numbers, lower quality, and did not contain subtypes that were missing in the poly(A) dataset. In this example, there was then a total of 455 clusters (406 tumor and 49 normal tissue classes respectively) of which 303 were non-overlapping independent terminal (leaf) nodes. In this context, overlapping generally means clusters that have common samples. For example, a parent and a child cluster along the hierarchy will have common samples (the child cluster is made of a subset of samples in the parent cluster) and can be considered overlapping.

In the example experiments, starting from Trimmed mean of M-values (TMM) normalized data, which already accounts for the skew introduced by extreme values across the population, the expression was standardized along genes to limit heteroscedasticity. Being additive, Shannon entropy cannot be naively measured on groups with different populations, and it requires enough samples to approximate a continuous distribution. In these experiments, this holds true for a good number of classes, but not for the smallest leaf nodes. Thus, a first approximation was performed on the expression distribution of every single gene independently with a fixed-bandwidth Gaussian kernel density estimation and then extraction of the probabilities for Shannon entropy from the estimated distribution on a 100,000 points grid. Higher resolution grids approach the limit of differential entropy and approximate the integral better, yet they only lead to marginal changes and increase the computational cost considerably. A mesh to 250,000 points led to a change in entropy of less than 2%, confirming that the choice was close to convergence. The entropy calculation was limited to a subset of more than 14,000 highly variant genes, by filtering those with both consistent low expression across samples and low entropy across all classes. The final values obtained for each gene and each class were divided by the median entropy of the normal tissues cohort class N000.

RNA-Seq expression data can be approximated by a negative binomial distribution, which accounts for overdispersion in its mean-variance relationship. The coefficient of variation can be used as a measure of mean-independent dispersion; however, it still relies on variance and thus can inherit the shortcomings when attempting to quantify transcriptional noise. The example experiments observed a fair correlation between entropy and mean expression across all groups (Pearson's r 0.68). A linear model can be fit on the entropy and the score adjusted to account for its dependency on the median expression. The coefficient of determination R² was 0.46, suggesting the mean-dependent component accounts for less than half the total entropy, and transcriptional noise within and across tumor types cannot be entirely explained by differences in expression levels alone. This adjusted entropy (S) was used in the example experiments.

### Classifier

When run on an example dataset of more than 13,000 tumour and healthy normal tissues, the recursive clustering algorithm performed by the optimization module 104 outputted an extensive hierarchy of more than 455 classes, 8 levels deep. While training a CNN classifier is a fully supervised operation, using these data-driven cluster classes as target allowed the classification module 108 to generally shed reliance on original labelling, a possible source of noise and errors.

In the example experiments, the resulting trained model was found to be markedly more accurate and robust than alternative classification methods; such as k-nearest neighbors (k-NN), which are affected by tears, deformations, and the partial loss of meaningful distances in the dimensionally-reduced space, and have limited flexibility when dealing with multiple tumor components.

TABLE I illustrates a summary of the parameters for each CNN in accordance with this example, where binary cross-entropy was used as the loss function and Adadelta was used as the optimizer:

TABLE I

| | CNN01 | CNN02 | CNN03 |
|---|---|---|---|
| Pre- 1dConv filters | 32 | N/A | N/A |
| Pre- 1dConv kernel | 4 | N/A | N/A |
| size | | | |
| Pre- 1dConv strides | 1 | N/A | N/A |
| # 1dConv layers | 1 | 3 | 2 |
| 1dConv filters | 81 | 192 | 204 |
| 1dConv kernel size | 2 | 4 | 4 |
| 1dConv strides | 1 | 2 | 1 |
| Pool strides | 2 | 2 | 2 |
| Pool size | 4 | 4 | 4 |
| # FC layers | 1 | 3 | 2 |
| # FC starting size | 1552 | 2048 | 2048 |
| Dropout percent | 0.4 | 0.4 | 0.4 |

In an example, Hyperopt was used to identify optimal architectures; it is a Bayesian hyperparameters optimization library based on a Tree-structured Parzen Estimator (TPE) approach. A micro-F1 (μF1) score was chosen as the objective function to guide the search. To this set of models, a number of alternative manually-tuned architectures were added. The manually-tuned architectures can follow the same structures, but with parameters that were set manually to test regions of the hyperparameters space that were not selected by the hyper-optimization. The latter can occasionally get stuck in regions local optima and miss alternative, but still valid, high-performing combinations. For example, the algorithm can select models with high-precision and low-recall. These extra models can help in subsequent selection steps.

In an example, all models included 1D convolutional (CV) layers followed by fully connected (FC) layers. The number of filters of CV layers was tuneable and shared across layers, and so was the kernel size. Each CV layer was followed by batch normalization and max pooling with fixed size 4 and stride 2. The size of hidden dense layers was also tuneable and halved at every successive layer, dropout was activated at parametrizable percentage. The loss function was binary cross-entropy. Adadelta was set as an optimizer with a starting learning rate of 0.001 and early stopping, The top scoring models among the pool of candidates were subject to a 5 randomized rounds of 5-fold cross validation to evaluate their performance under different conditions of training and validation. The splitting into train and test sets was stratified, to assure a proportional coverage for every class and early stopping after three epochs was activated at convergence of the loss function to avoid overfitting. Five candidate models were then selected according to their different performances on a set of scores including macro-F1 (MF1) and macro-precision-recall area under the curve (MAUCPR). The classifier used by the classification module 108 in this example used an ensemble average of a subset of these models. Out of different combinations of models and averaging methods, an unweighted arithmetic mean of three can be used. The employment of an ensemble classifier can lead to a marginal improvement in most scores, while limiting the shortcomings of each single model and adding robustness to the final predictions. However, in other cases, a single model can be used. The classification module 108 trains the model(s) using available samples, with an adequate number of epochs to avoid overfitting for each separate case.

The example experiments compared the results of the transcriptional classifier performed by the classification module 108 to a DNA methylation-based classifier, for a set of CNS tumours. Previously, 252 high-risk pediatric cancers were profiled through multiple sequencing technologies. 63 of these are CNS tumours with data from both DNA methylation and RNA sequencing and can be directly compared.

Following a manual curation, the methylation classifier matched these tumours to their presenting clinical diagnosis in 86% of the cases. The remaining 14% were either matched to a wrong subtype but within the correct parent family, or do not match the expected subtype. The two classifiers agree in almost all these cases, within the limits of tumour types available in the respective reference datasets and complement each other in the few exceptions.

Among the 8% of samples matched only to the parent family according to DNA methylation, the transcription-based classifier performed by the classification module 108 could correctly identify the subtype of 3 samples: a medulloblastoma, called as retinoblastoma by DNA methylation; a low-grade glioma, instead of a high-grade glioma; an ependymoma, whose methylation profile was reflecting immune infiltration. In two cases, the classifiers were in agreement, in spite of a mismatch with respect to the pathologist diagnosis: a IDH wild-type glioma and a medulloblastoma of the G3 subtype. Finally, there are three cases in which the transcriptional classifier fell short, where the DNA methylation matched the correct subtype: an ependymoma, which was not recognized by the classification module 108 due to low purity and high immune infiltration, and two atypical teratoid rhabdoid tumours, a subtype which is absent in the RNASeq reference.

Evidently, the classification module 108 can provide highly accurate predictions in the most complex of cases.

According to example experiments, the classifier of the classification module 108 is able to maintain substantial performance in classifying samples down to a few million reads with a greater than 80% hierarchical rity (a metric used to evaluate accuracy and similarity in the prediction across samples while accounting for the hierarchical structure of the predicted classes), when comparing these same samples with predictions obtained with more than a hundred million reads.

### Hierarchical Similarity

In some cases, to evaluate the accuracy of predictions, the classification module can use a hierarchical similarity score (H), which is a union/intersection score based on the Graph Information Content (SimGIC) that measures the proximity of two points along the class tree while accounting for its structure and populations.

$$H ( {v}_{1} , {v}_{2} ) = 1 - {Δ}_{u / i} ( {v}_{1} , {v}_{2} ) = \frac{{\sum }_{n ∈ nodes ( {v}_{1} ) ⋂ nodes ( {v}_{2} )} w ( n )}{{\sum }_{n ∈ nodes ( {v}_{1} ) ⋃ nodes ( {v}_{2} )} w ( n )} = \frac{{\sum }_{n ∈ nodes ( \vec{1} )} w ( n ) min ( {v}_{1} ( n ) , {v}_{2} ( n ) )}{{\sum }_{n ∈ nodes ( \vec{1} )} w ( n ) max ( {v}_{1} ( n ) , {v}_{2} ( n ) )}$$

where v₁, v₂ are the membership assignment probability vectors of two samples, nodes (vₓ) is the list of nodes or classes activated in such vectors, nodes(\vec{1}) is the list of all nodes and w(n) are the nodes' weights. These are calculated as information content, that is the probability of a sample falling into the lower node connected to the edge, which can be approximated to the class frequency of observations in the training dataset.

$${W}_{SimGIC} ( n ) = - log p ( n )$$

A partial hierarchical similarity score (n) can also be used, which only looks at the branches active in the ground truth, while disregarding false positives.

$$η ( {v}_{1} , {v}_{2} ) = 1 - {δ}_{u / i} ( {v}_{1} , {v}_{2} ) = \frac{{\sum }_{n ∈ nodes ( {v}_{1} ) ⋂ nodes ( {v}_{2} )} w ( n )}{{\sum }_{n ∈ nodes ( {v}_{1} )} w ( n )} = \frac{{\sum }_{n ∈ nodes ( \vec{1} )} w ( n ) min ( {v}_{1} ( n ) , {v}_{2} ( n ) )}{{\sum }_{n ∈ nodes ( \vec{1} )} w ( n ) {v}_{1} ( n )}$$

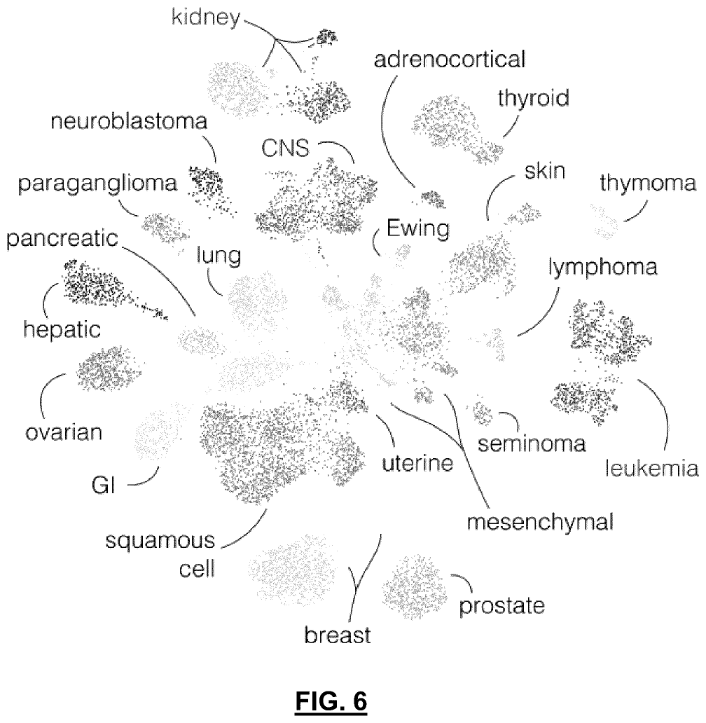

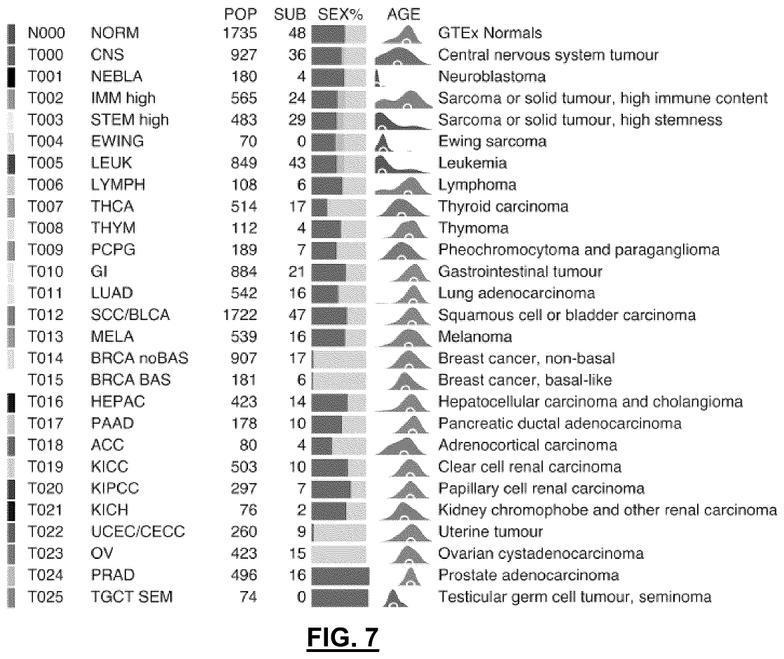

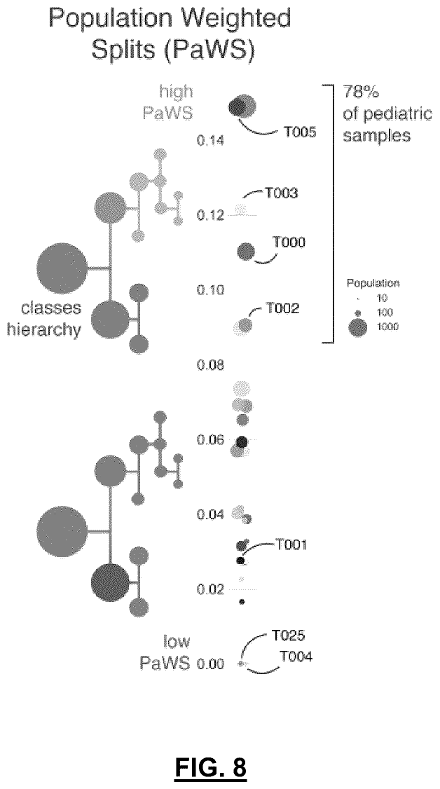

The example experiments verified the advantages of the present embodiments. 13,313 tumor and non-neoplastic samples were divided into 455 distinct classes, arranged across 8 hierarchical levels, as illustrated in FIGS. 6 and 7. There were 26 main tumor types at the top-most level, which were then further divided into up to 48 subtypes each. To better understand the structure of these trees, a score was used to measure the relative size of offspring branching along the hierarchy tree, called the Population Weighted Splits (PaWS), as illustrated in FIG. 8. Four main tumor types with the highest PaWS scores (i.e., deepest branching), encompassed a large proportion of the whole cohort. Together, the pan-leukaemia group (T005 LEUK, n=849, PaWS=0.15), squamous cell cancers (T012 SCC/BLCA, n=1,722, PaWS=0.15), central nervous system tumors (T000 CNS n=927, PaWS=0.11), and sarcomas (T002 IMM high n=5657 PaWS=0.09 and T003 STEM high n=483, PaWS=0.12) accounted for nearly 39% of all available tumor samples. 192 tumor subtypes descend from these 5 top-level tumor types.

### Dataset

The example experiments used a reference dataset that included the UCSC Treehouse Childhood Cancer Initiative Compendium (v9, March 2019) to build a hierarchy of subtypes and train the ensemble CNN classifier. This cohort included 11,750 tumor samples from TCGA, TARGET and other contributing institutions, prepared with either Poly(A) selection or Ribo-depletion. Gene expression counts from the STAR+RSEM toil-RNASeq pipeline of samples in the compendium are publicly available and cover more than 58,000 genes, raw or normalized by log 2 TPM. To expand the pediatric reference, 313 further samples from St. Jude Children's Hospital Pediatric Cancer Genome Project (PCGP) were also added and run through the same pipeline after filtering them by alignment quality. While building the subtypes hierarchy, samples were removed with particularly low purity, but kept them to boost the ensemble CNN training. 1,735 normal tissue samples with the best coverage and quality scores from 51 different organ sites from the GTEx project were also added to the dataset. To avoid degradation in the output due to the possible presence of tumor samples with normal contamination within the Treehouse cohort, the tumor and normal datasets were kept separate at the first stage of the clustering and merged later as separate branches of the classification hierarchy. Genes mapping to non-coding sections of the RNA were removed. Among these remaining, only genes with high variability, accounting for 99% of the cumulative variance on the full cohort, were kept. This reduced the feature space to 18,010 functional genes and pseudogenes allowing speeding up of the rest of the analysis with a negligible loss of information. log₂ TPM-normalized counts were used for clustering, classification, and map projections. For differential expression analysis, TMM normalization the negative binomial generalized log-linear model fitting from EdgeR were used. Gene sets enrichment analysis (GSEA) was carried out with EGSEA and gseapy. The Mann-Whitney U test (MWU) was used when evaluating significance in comparing distributions. These include single sample gsea scores between adult and pediatric samples, to compare pathway expression.

In some of the example experiments, the classification model was trained on a dataset of poly(A)-seq samples from fresh-frozen (FF) tissue. To evaluate its 9 generalizability to alternative library preparation techniques, its performance was tested on a set of 247 samples from the ThreeHouse childhood cancer initiative compendium treated with ribosomal depletion. The closest possible tumour class to the provided diagnostic label was set as ground truth. Tumour subtypes that were absent in the reference were, in some cases, removed; however, tumours lacking a matching class but with a consistent population in the reference cohort were included. As an example, gastrointestinal stromal tumours, which are consistently found in the T078 SARC EPITH/KIT class but lack for now their own subgroup due to a limited population (n=6) were included and so were samples with myofibromatosis. The classifier can still identify the correct tumor type, albeit in some cases with lower confidence.

### Childhood Cancers

In general approaches, tumors from a similar tissue of origin are typically co-clustered at the top-level. However, new factors emerged as drivers of transcriptional difference as one looks within trees or explores their structure. Age is an important factor—the distribution of cancer types differed between adult and childhood cancer. 85% of pediatric cancers belonged to only 6 out of 27 trees, but these are more likely to involve deep subtypes (mean PaWS 0.83 vs 0.50, mean number of offspring nodes 22.6 and 12.2), many of represent novel cancer subtypes. Similarly, non-neoplastic samples were first grouped by tissue of origin, yet occasionally, the transcriptional stratification transcended the organ of origin.

To further define the transcriptional subtypes of childhood cancer, the example experiments annotated in depth 162 tumor clusters, including every majority pediatric class identified by the optimization module 104, noting their associated changes in survival, age, sex, and the underlying genomic alterations where possible, as well as key genes differentiating these clusters from their adult counterparts.

Having defined childhood-specific cancer subtypes, the example experiments investigated their internal differences in gene expression. Expression fluctuations at the level of individual genes were investigated among tumors whose overall transcriptional profiles were similar (i.e., expression changes of the same genes among tumors in the same cluster). These fluctuations were quantified using Shannon Entropy(S) a concept borrowed from thermodynamics and information theory, where in this context, can be thought of as the “transcriptional disorder” of tumor subtypes.

Overall, non-neoplastic tissue was determined to be less disordered than cancers. Normal cells appear to allow for a narrow range of expression for each gene, whereas tumor types can tolerate more variation in gene expression while still maintaining a characteristic expression profile (11% higher entropy on average at the first hierarchy level). The same was true when comparing tumor clusters to their closest matching non-neoplastic types (10% higher S on average); not only did tumor subtypes have a significant increase in transcriptional disorder compared to their closest normal equivalent but there was a positive correlation between most of them (Pearson 0.56, p-val=2.29e-02). This suggests that a tumor's transcriptional variability may to some degree be predetermined by its tissue of origin.

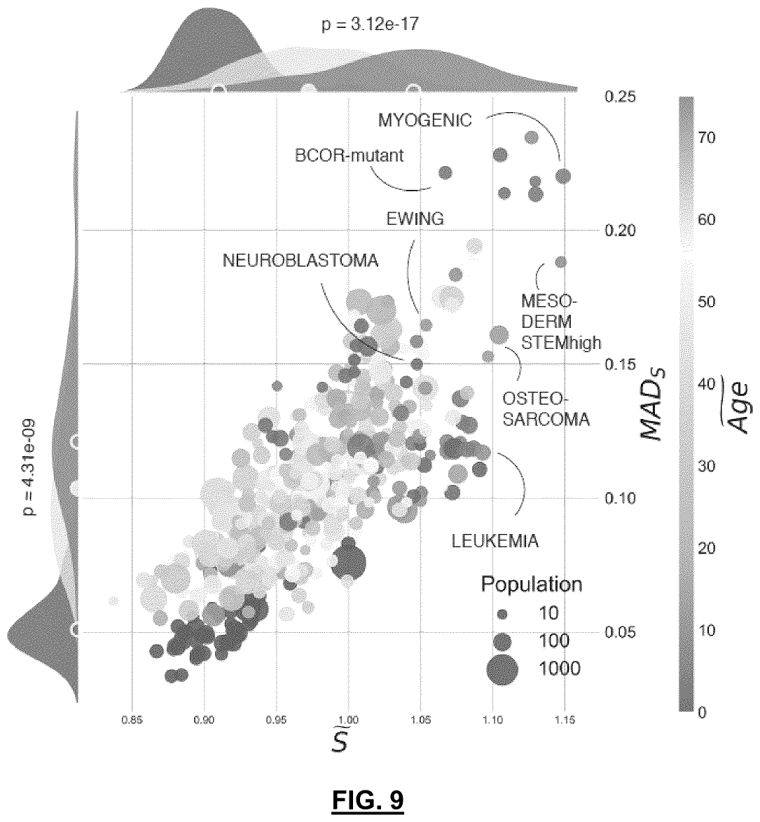

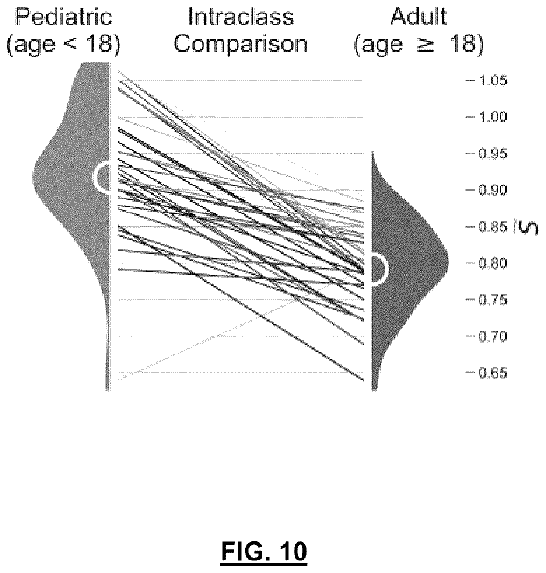

Childhood cancers typically have lower somatic mutation burdens than adult tumors. As there are fewer mutations potentiating gene expression changes, one would expect a less noisy transcriptome in pediatric cancer. In contrast, when looking within well circumscribed tumor classes, the example experiments found significantly higher transcriptional disorder in childhood cancer, as illustrated in FIG. 9. This was true across cancer types (<18 y.o. vs >=18 y.o. Mann-Whitney U test, MWU, p-val=3.12e-17) as well as when comparing childhood to adult brain cancer (n.s.), leukemia (p-val=1.79e-06), and sarcoma (p-val=2.71e-04). This discrepancy holds true even after removing sampling bias by maintaining only classes within the population interquartile (p-val=1.18e-05). It is further corroborated by an intra-class comparison in all but one tumor types pediatric cancers had higher disorder than their adult equivalent, as illustrated in FIG. 10.

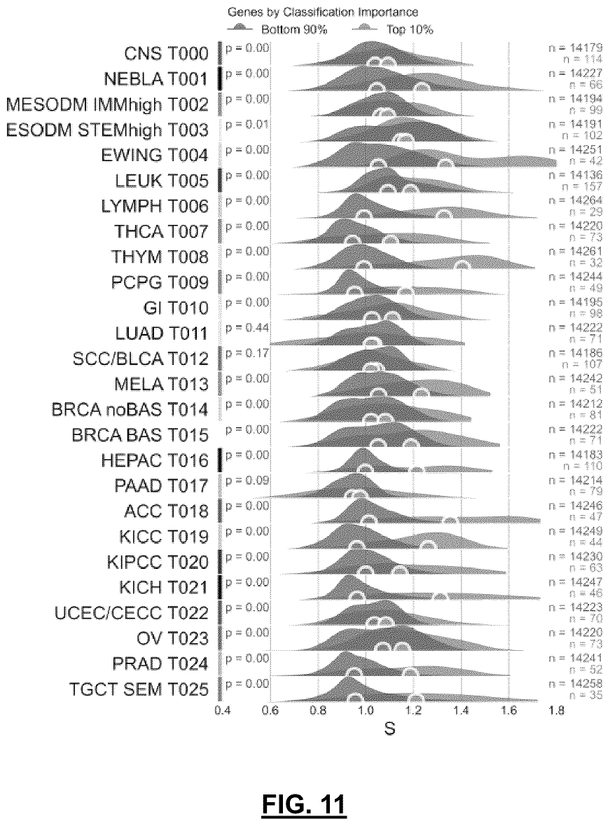

The example experiments examined whether the excessive transcriptional disorder seen in childhood cancer involved a subset of expressed genes or large parts of the transcriptome. This can be quantified by the median absolute deviation (MAD) of the per-gene entropy distributions: small values mean most genes are similarly entropic, high values mean their disorder level can vary widely. In childhood tumor types the transcriptional disorder is broader, impacting different genes to different degrees, their MAD is significantly higher than in adult tumors (MWU p-val 4.31e-09). The example experiments observed that the most disordered genes, represent marker lesions localized to a small portion of the genome, and are remarkably specific to each subtype. To support this, a feature importance extraction approach (DeepLIFT), was used to rank the genes used as input for the ensemble CNN by their relevance in identifying each tumor type. In most tumor types, the top 10% cumulative importance genes are also those with the highest entropy, as illustrated in FIG. 11. These corresponded to disease defining pathways, as when ranking genes by importance score, it was observed that an enrichment of marker lesion genes and gene sets (e.g. Ewing family genes and EWSR1-FL1 targets²³⁻²⁶ in T005 Ewings; neuronal development and differentiation in T035 CNS BCOR, GSEA p-val <0.001, FL1 and BCOR <top 1% most important genes respectively). The exceptions were lung, pancreas and squamous cell tumors. Compared to adult malignancies, childhood cancers are mostly transcriptionally distinct, forming unique subtypes. Yet, within these subtypes of childhood cancer, there is remarkable plasticity among the disease-defining genes.

### Sarcoma

Ewing sarcoma (ES) provides an example of the uniqueness of pediatric cancers as identified by the present embodiments. ES is found in a unique cluster, separate from the sarcomas or indeed any other cancer type. ES is one of 26 top-level tumor types and one of only three to have no descendants. These unique transcriptional features can be used as a straightforward diagnostic test. It was determined that up to 12% (9/80) of ES tumors by standard pathology may be misdiagnosed CIC- or BCOR-driven sarcomas.

Sarcomas, cancers of the bone and soft tissue of which there are >150 varieties, are proportionately more common in childhood and are notoriously difficult to diagnose or subtype. The example experiments were able to identify 55 sarcoma and mesodermal solid tumor clusters, including 37 subtypes, most of which either contain a known fusion or are derived from a common tissue. Using this approach, one can clearly distinguish, osteosarcoma (T068), leiomyosarcoma (T067), fusion-positive and negative rhabdomyosarcomas (T094 and T093), synovial sarcomas (T100) and others. Importantly, other cancers that are thought to derive from the mesoderm, such as mesothelioma (T070), Wilms tumors (T092), choroid plexus carcinomas (T102), and testicular non-seminoma germ cell tumors (T101 and T105) also clustered with sarcomas, while Ewing sarcoma (T005) did not. Overall, the transcriptional contribution from the tissue of origin appears to be greater in sarcoma than carcinoma, as reflected by the greater number of clusters at the first levels of the hierarchy, as illustrated in FIG. 6.

In the example experiments, the sarcomas were primarily constrained to just two mesodermal superclasses. The first (T002) was comprised of osteosarcomas, leiomyosarcomas, mesotheliomas and a mix of less differentiated sarcomas. The second (T003) assembled alveolar and embryonal rhabdomyosarcomas, synovial sarcomas, testicular germ cell tumors and Wilms. T002 and T003 were then subtyped into 24 and 29 specific mesodermal subclasses. Both mesodermal superclasses expressed epithelial to mesenchymal transition (EMT) markers as expected, but otherwise had remarkably divergent gene expression programs. On the one hand, T002 sarcomas displayed high immune cell activation, with enrichment for pathways 26 indicating a robust host immune response (fdr<0.001). Consistent with this, T002 had higher leukocyte content and proportion of M2 macrophages (and M1 macrophages, to a lesser degree), along with high overall stromal content, while the ratio of lymphocytes to myelocytes was no different. Thus, T002 sarcomas display a significant imbalance of immune cell types indicating a highly immunogenic tumor microenvironment.

Exemplary of this active immune profile is T080 SARC IMMUNE, a small subclass of mixed solid tumors characterized by high leukocyte fraction, a high portion of M2 macrophages and CD8+ cells. It is mostly composed of dedifferentiated liposarcomas (11/30) and undifferentiated pleomorphic sarcomas (9/30). Despite the variety of tumor subtypes within this class, they show a noteworthy homogeneity of expression, as reflected by the low entropy and their definition during clusters search, possibly due to the preponderance of an immune transcriptional signal and the lack of idiosyncratic profiles because of their undifferentiation.

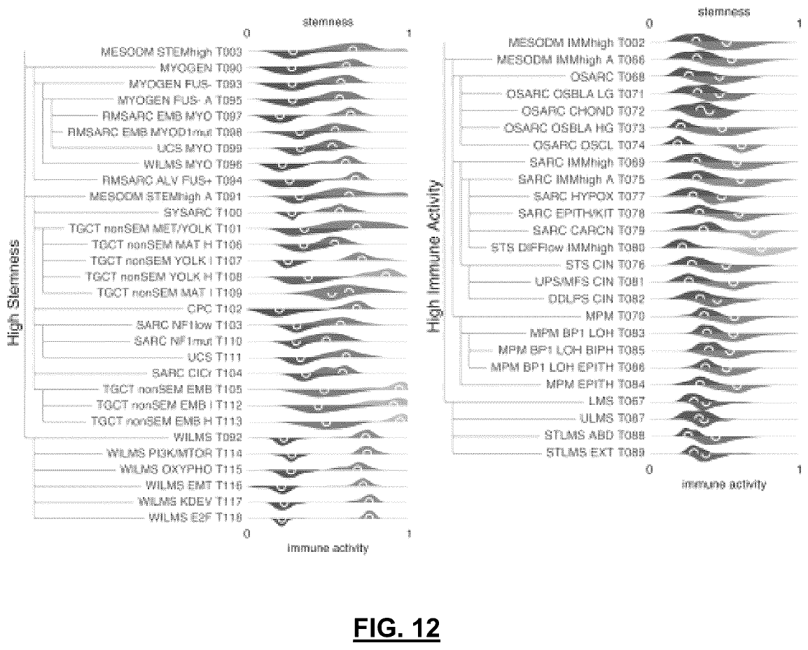

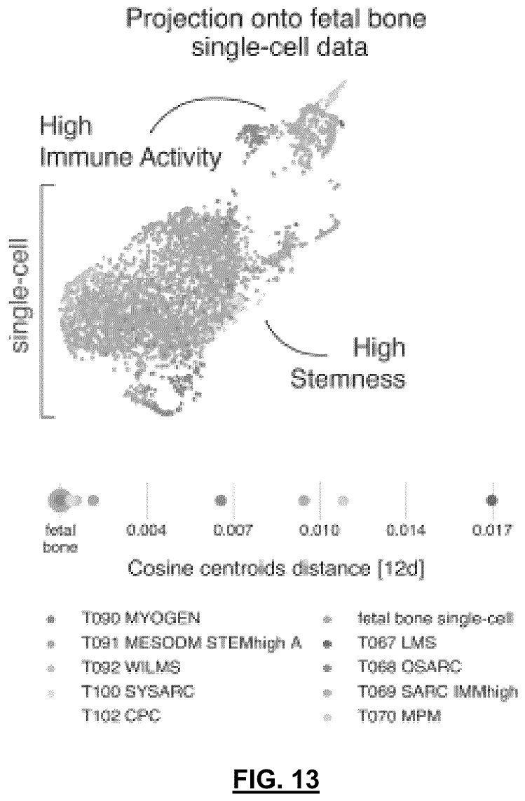

The second mesodermal superclass (T003) involved low immune activity and remarkably high markers of “stemness”. Among the gene sets most differentially expressed, it was found that stemness markers were significantly enriched in T003 STEM high (p-val<0.001). The stemness level of samples were measured within T003 using three independent methods, cytoTRACE, mRNAsi and ssGSEA signature. There was a negligible relationship between stemness and tumor purity (Pearson's correlation 0.14, p-val=8.83e-04 on TGCA data only). All tools concordantly show a statistically significant enrichment (MWU p-val=5.41e-165, as illustrated in FIG. 12) of stemness in T003, although differences are observed within subtypes as expected. This suggested that T003 represented a class of mesodermal cancers of embryonic origin. This notion is supported by the inclusion of rhabdomyosarcomas and germ cell tumors in T003. To further explore this, tissue was obtained from a fetal sample estimated to be 56 days in postconceptual age, sequenced 37,490 cells, and then compared their gene expression profiles to that of the bulk sequenced sarcomas. Overall, the T003 class of cancers was more similar to fetal cells, with some subtypes within the class, even clustering immediately adjacent to in utero cells, supporting their early origins, as illustrated in FIGS. 13. Taken together, these results support the idea that T002 (“IMM high”) is a class of mature malignancies characterized by high stromal content, vascularization, and an active immune profile. This contrasts with T003 (“STEM high”), which includes sarcomas with a more immature phenotype, possibly reflecting their embryonic origin. It is likely that their common mesodermal lineage brings these disparate solid tumors together while keeping them apart from the rest of cancer.

### Other Cancers

In addition to sarcomas and other mesenchymal tumors, the example experiments illustrated how the system 100 was able to identify clusters for most major types of pediatric leukemia, brain tumors and solid cancers. For nearly every recognized pathological classification of pediatric cancer, there was a corresponding transcriptional cluster. For instance, in brain cancers one can differentiate molecular subtypes of medulloblastoma (T027), 1p/19q codel gliomas (T044) as well as those with and without IDH1 mutations (T030 and T29), ependymomas (T032), among others. Within the leukemias classes, almost all of which are pediatric, one can differentiate BCR-ABL1 positive ALL, as well as those with Ph-like variants (T127, T137, T139 and its child classes), distinct subclusters driven by fusions in CBFB-MYH11 (T145), PML-RARA (T147), TCF3-PBX1 (T135), RBM15-MKL1 (T515), as well as AML with KMT2A internal tandem duplications (T153), KMT2A rearrangements (T159) and many additional leukemia subtypes (Fig. S8). For rarer subtypes, the example experiments showed evidence for emerging clusters that will likely be further subtyped when more samples are available. Thus, both well established and novel subtypes of childhood cancer can be assessed using the transcriptome-based approach of the present embodiments.

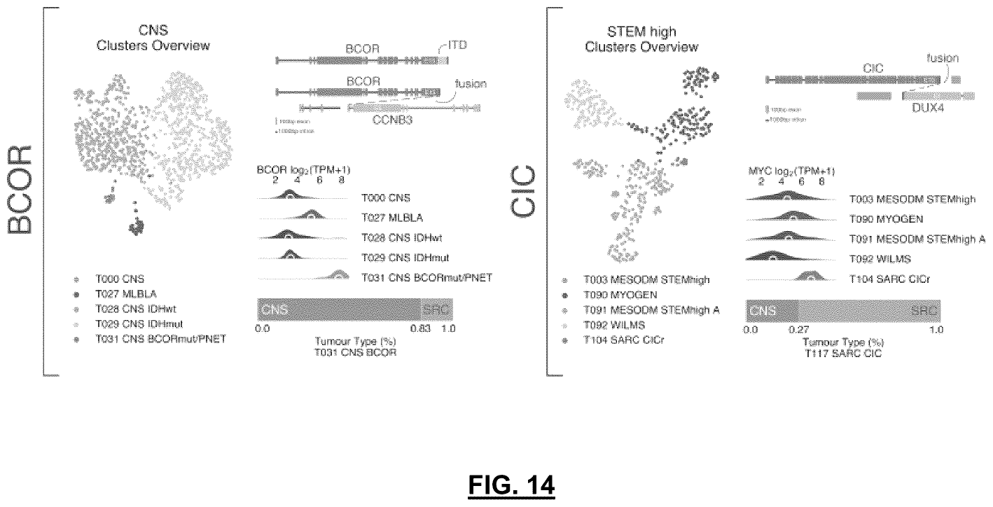

Childhood tumors of different histotypes were occasionally brought together into a single cluster by the system 100, indicating unexpected, shared gene expression programs or a common cell of origin. For instance, within the hierarchy of brain tumors, is a relatively small (n=12) but highly specific cluster of young childhood tumors (average age 4.5 yrs.). Of note, this cluster (T031) is made up of both CNS and extra-CNS cancers with BCOR-associated gene expression programs. Validating this annotation, all but one sample contained BCOR alterations, including fusions, partial deletions and internal tandem duplications. Similarly, within the sarcoma branch, one finds a new class of round blue cell tumors of mixed origin, including both sarcomas and brain tumors, with an average age of only 12 years (T117). All have expression patterns reflective of CIC-DUX4 fusions, as illustrated in FIG. 14. While these tumors are notoriously difficult to diagnose and have been sometimes classified as Ewing sarcomas, the example experiments illustrate the notion that the system 100 was able to determine they are in fact a distinct entity, independent of the anatomic location in which they arise.

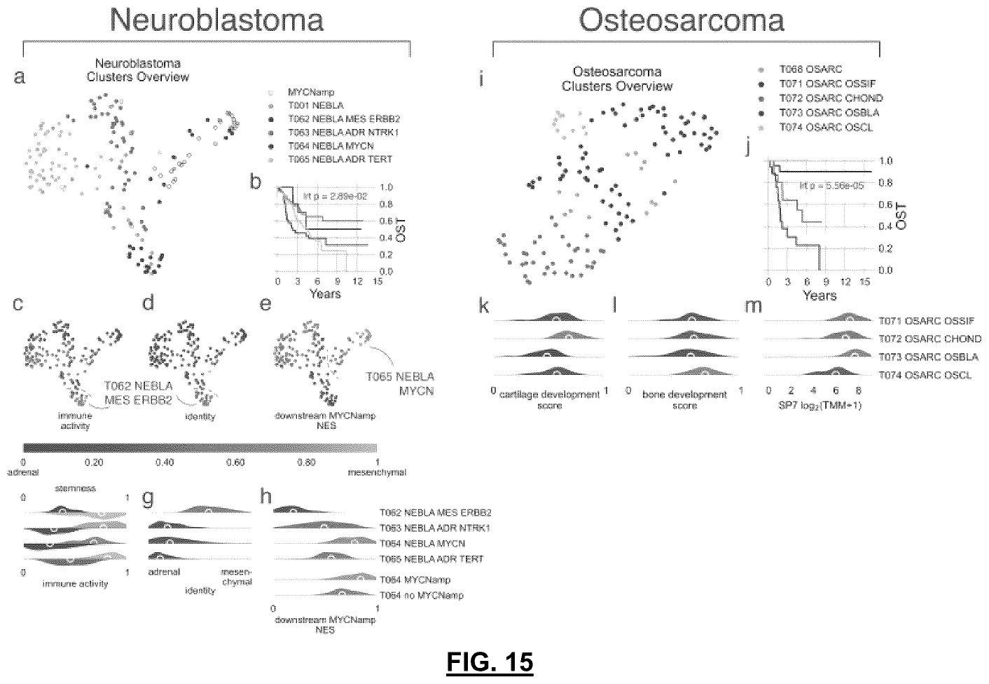

The example experiments also found four subtypes of neuroblastoma, the most common childhood extracranial solid tumor, as illustrated in FIG. 15. These subtypes, which overlap with previously-reported clusters, have significant differences in immune activity, differentiation level and survival. Furthermore, their effect on survival is independent of COG risk group and stage. Named based on the expression of previously established marker genes, ERBB2 (T062), NTRK1 (T063), MYCN (T064) and TERT, these subtypes may be rooted in the tumor's lineage. The ERBB2-overexpressing subtype are highly differentiated, with high immune activity, and reflected a neural crest cell/mesenchymal identity. Conversely, the TERT subtype is associated with a sympathoadrenal identity and has the highest level of stemness. These subtypes were only partially correlated with the established COG risk groups, which are primarily based on histology and MYCN copy number. Of the 35 patients in the MYCN expression class of the example experiments, 25% (9/35) were not previously identified as MYCN amplified by standard testing, but still maintained significant enrichment of downstream MYCN amplification pathways. That is, these neuroblastoma patients had the transcriptional fingerprint of activated MYCN, yet would have been misclassified by conventional cytogenetics.

Osteosarcoma, the most common bone tumor of childhood, was also readily subtyped by the system 100 in the example experiments, as illustrated in FIG. 15. It is typically stratified using histology and tumor location. In the example experiments, four osteosarcoma subtypes were identified, which separate from one another by different levels of bone and cartilage development genes. Critically, the four subtypes of osteosarcoma also lead to significant differences in prognosis (Irt p-val=5.56e-05). Among these there is a class characterized by osteoclast differentiation with good prognosis (T074), a second high-survival rate class with enrichment of osteoblast differentiation, direct ossification and cancer testis antigens (T071), a chondroblastic osteosarcoma group with low-to-intermediate survival rate (T073) and a bona-fide osteoblastic osteosarcoma class, presenting the overall worst survival curve (T072). Going beyond the characterization of histotypes and cell development, whole transcriptome profiling can unlock stratification with prognostic utility.

### New Tumors

Having determined that the system 100 can be used as a diagnostic and

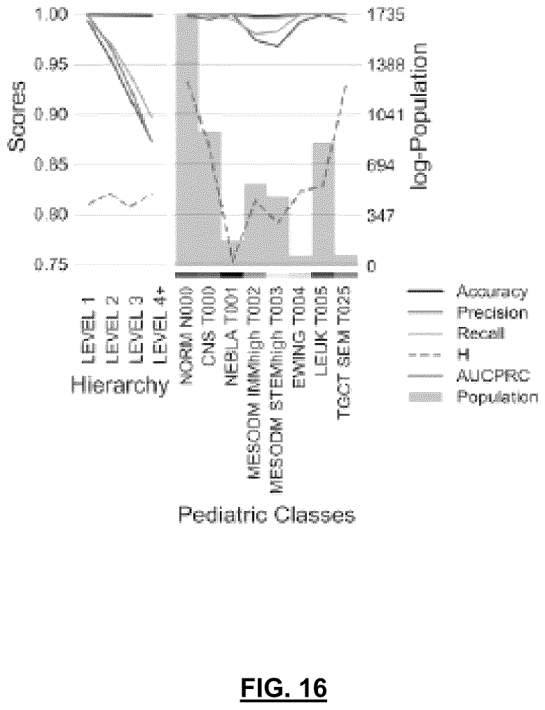

prognostic aid for childhood cancer, the example experiments also validated the use of the neural network to classify new tumors from ongoing patients. The ensemble convolutional neural networks used by the classification module 108 to prospectively classify new patients' tumors was determined to outperform alternatives and reached >0.99 mean AUCPR across major pediatric malignancies, while maintaining excellent performance even for minor subtypes found deep in the hierarchy; as illustrated in FIG. 16. Tumor-derived RNA from childhood cancer patients enrolled in an ongoing precision medicine program were sequenced (163 tumors from 132 patients). The ensemble was 9 applied to this held-out cohort and tumor classifications were compared to the original diagnosis. With a median age of seven years, the patients analyzed were broadly representative of the hard-to-cure tumors seen at most large childhood cancer centres: 44% (72/163) were either relapsed, refractory or metastatic disease; and 60% (97/163) were post-therapy. Across the cohort, the tumor classification performed by the system was concordant with the Pathologists' diagnosis for 66% of the cases, as illustrated in FIG. 16. This included both molecularly-defined cancers (e.g., Ewing sarcoma) as well as those defined by specific immunohistochemistry/cell morphology markers.

The system's tumor classification was concordant with the pathologists' diagnosis for 65.6% of the cases. In an additional 16.6% of cases, the system 100 confirmed the diagnosis even in the absence of a corresponding tumor type in the reference set, by using a comparison of their class assignment to similarly labelled samples in both the reference and the validation cohort. In the example experiments, the majority tumour class assignment for each sample was compared with the original diagnosis provided by the pathologist. It was assessed whether this assignment corresponded to a tumour class that matched the provided diagnosis or to a tumour subtype in the proximity to the matching diagnosis in the hierarchy. In this way, if the subtype does not match but belongs to the same family; for example, if a myeloid leukemia is assigned to just the leukemia parent or a different type of leukemia. For samples that were presented with a diagnosis that was absent from the reference cohort, the result was compared with samples with the same diagnosis. If all samples belonging to this group were consistently assigned to a certain group, and this group was biologically close to the missing diagnosis, they were noted as a match without reference.

The ability to work with tumor types outside of the training domain is crucial in a clinical setting, as it expands the reach of the classifier to include rare neoplasms, for which it is unfeasible to gather a sufficient training dataset. Of note, with the help of the system 100's classifier, the diagnosis was updated for 11 cases (7% of the cohort). This included four BCOR-rearranged sarcomas, a clear-cell sarcoma of the kidney harbouring a BCOR-ITD, two infant lymphoblastic leukemias with MLL partial tandem duplication, and two megakaryoblastic leukemias with sarcomatous components. Altogether, the system's classification was correct, in that it either matched or refined the pathologists' diagnosis, for 88.9% of cases.

Because the outputted prediction probabilities are multi-label (where samples can be assigned to more than one class), the system 100 was able to identify samples with a high degree of contamination by non-tumor tissue. Normal tissue expression was the dominant profile in 4% of the samples and was present to a lesser degree in an additional 4% of tumors. The example experiments confirmed the diagnosis of 89% (16/18) of the samples with <50% tumor purity. For the remaining samples, 9% showed signs of necrosis or other quality-related issues. And 6 samples (4%) remained inconclusive, even without any obvious quality issues, due to the current lack of a comparable match in the reference class hierarchy.

To evaluate the robustness of the system 100's tumor predictions over time, multi-time points samples (e.g., primary-relapse and/or primary-metastasis pairs) were sampled. 21 patients had >1 tumor sequenced (between two to four tumors, excluding two patients with multiple primaries). 86% of these multi-sample cases maintained a consistent class assignment over time, as illustrated in FIG. 15. The exceptions to this included two Wilms tumors with evidence for normal contamination (0074 and 0120) as well a rare type SMARCB1-associated tumor that is currently absent from the reference set (0154). Taken together, the tumor type predictions are highly concordant with those of pathology, can help to clarify ambiguous diagnoses, and stay consistent across time even as the tumors evolve.

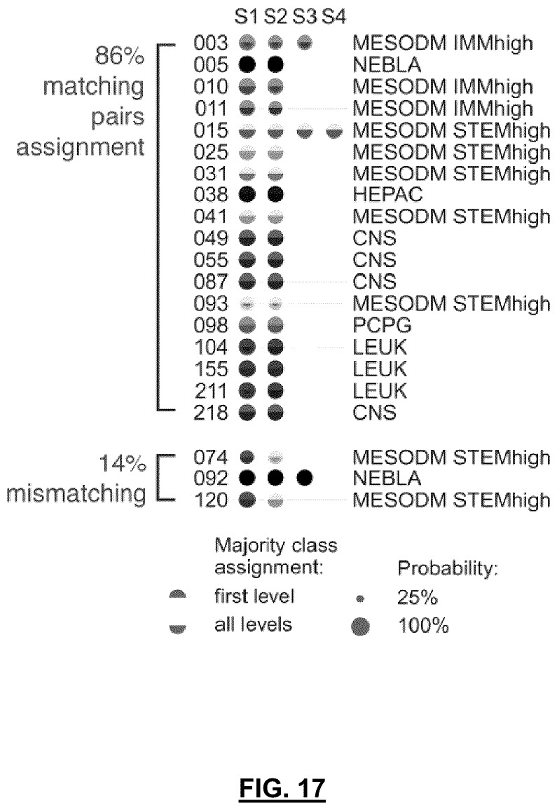

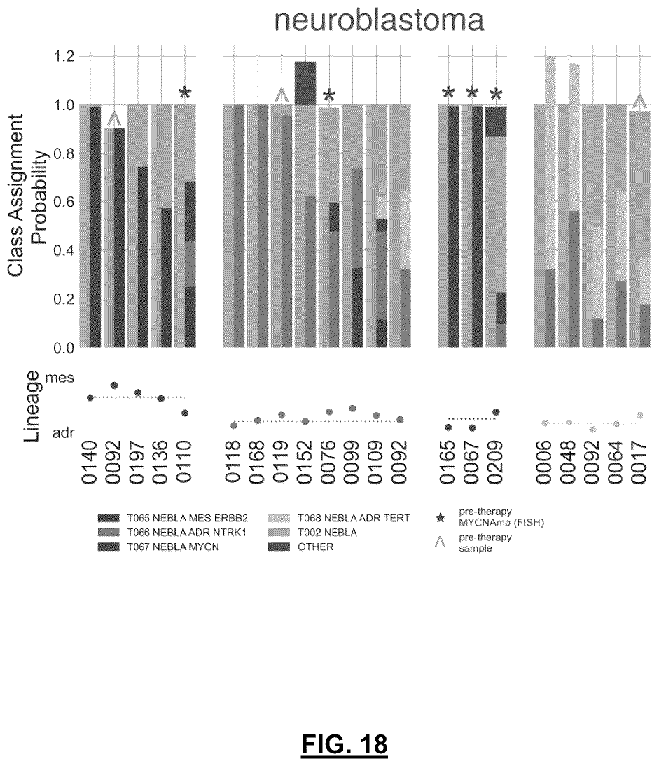

From this temporal analysis, the only tumor to dramatically switch its transcriptional profile at relapse was a neuroblastoma, as illustrated in FIG. 17. To explore this further, the variability in the class assignment in all neuroblastomas (including those with only one time point sequenced) was measured and compared this to all other cancer types. It was found that individual neuroblastomas expressed multiple transcriptional programs at the same, as illustrated in FIG. 17. More than half of the available neuroblastoma samples (11/21) were in fact comprised of more than one subtype (with >2% confidence). Consistent with this, neuroblastomas that had been clinically subtyped as MYCN positive at diagnosis displayed a highly variable MYCN signature at relapse (subtype T077). The highly heterogeneous assignment of neuroblastoma subtypes seems to be unique among well-differentiated tumors. For instance, in contrast, all but one of the sequenced osteosarcomas were assigned to a unique subtype, as illustrated in FIG. 18. Neuroblastomas are known to maintain distinct cell states. The example experiments indicate that the system 100 is able to observe neuroblastomas' lineage plasticity and accurately quantify in vivo without using single cell analysis.

As evidenced in the example experiments, having quantified unique transcriptional features of pediatric cancers, the present inventors developed the system 100 as a diagnostic tool for childhood cancer, and cancer more broadly. Using an ensemble convolutional neural network trained on 455 transcriptional classes, the system 100 was able to match or refine the pathologists' diagnosis for 89% of ongoing patients. The system 100 does not necessarily rely on tumor site, morphology or immunophenotype and can accurately classify ˜90% of childhood cancers using a very small number of reads, as illustrated in FIG. 4. In addition to clarifying their diagnosis, the system 100 has prognostic utility, for instance by uncovering four types of osteosarcoma with clear differences in survival. Instead of giving each tumor a single discrete label, the multiclass models used by the system 100 can reveal simultaneous expression of more than one subtype within a bulk tumor. This was the case for >50% neuroblastomas that harboured more than one simultaneous subtype, even switching lineages after therapy.

### Benign and Malignant Neoplasm

Example experiments of the method 2000 suggest genes in identified groups have been selected by the GBoost models as relevant to distinguish the groups under investigation. From the first machine learning model classifier, the system 100 is able to extract, for example, immune activity, interleukin and integrin signaling, and markers of thyroid carcinoma. From the second machine learning model classifier, the system 100 is able to extract, for example, RAS and ESR1 pathways, cell adhesion, and locomotion and metabolic pathways. From the third machine learning model classifier, the system 100 is able to extract, for example, mTOR, GTPase and cell cycle. By leveraging the information from a substantial portion of the transcriptome (for example, 18,010 protein-coding genes and pseudogenes), rather than a manually curated set of genes, the first machine learning model classifier can reach, in an example, precision, sensitivity, specificity and NPV all above 99%.

The present inventors conducted example experiments to verify the advantages provided by the method 2000. The dataset used in the example experiments was composed of 512 papillary thyroid carcinoma samples from TCGA and 2 samples from participating institutions. 37 normal thyroid samples from healthy donors were obtained. A further 262 samples obtained, including both papillary and follicular thyroid carcinomas; 26 of these were labelled as benign tumours and 81 as adjacent normals from each of these subtypes. 47 pediatric tumour samples of which 18 are benign were also added.

Raw and log 2 normalized TPM expression counts were obtained from the STAR+RSEM toil-RNASeq pipeline and preprocessed. This provided 18,010 of the most variant protein-coding genes and pseudogenes. Recursive clustering was carried out, as described herein. Truncated Single Value Decomposition (tSVD) was used as low-information features removal, Differential Evolution was set as an optimization algorithm, Louvain community detection as a clustering algorithm, cosine was used as metric for both dimensionality reduction and clustering, dynamic mesh was activated with default parameters, and a 12-dimensions target space was selected.

TMM normalization and differential expression comparisons were carried out with edgeR, while single sample gene sets enrichment analysis (ssGSEA) was run with GSEApy on TPM normalized counts, after which the results were standardized. Disregulated gene sets were reported only if the FDR was below 0.05 unless stated otherwise.

When comparing populations, a two-sided Mann-Whitney U test (MW) test was applied. The Fisher's exact test (FET) was used for contingency tables and the χ² test for tables larger than 2×2. P-values were reported where appropriate and adjusted for multiple testing with a two-stages Benjamini-Hochberg if more than one comparison was carried out.

Immune activity and stemness of the test samples were tested by building normalized scores, by mixing leukocyte content fraction where available, and by averaging stemness scores from CytoTRACE, mRNAsi, and a gene sets-based score. A third score, quantifying tumour invasiveness in a similar manner, was determined by averaging the enrichment score of a list of curated gene sets.

In the example experiments, Gradient Boost models were built with XGBoost and Python; and hyperparameters optimization was carried out with Hyperopt and Ray. The models were trained on TMM normalized data, their input features were reduced with tSVD by keeping 0.999 of the total variance, and standardized. A randomized 5×5-fold validation was carried out to evaluate the performance of each model. Features importance scores were extracted from each model. Weight and gain were multiplied for each feature and then averaged across all 5×5-fold models produced. Gene sets enrichment analysis was performed on the resulting score.

The data set for the example experiments contained 860 samples: malignant and benign tumours, healthy and tumour-adjacent normal tissue (1a). Multi-scale clustering produced a tree of 81 classes with at least 10 samples each. For speed and simplicity, the example experiments limited annotation to the first 4 layers of the hierarchy, which include 28 classes. While there was no observed correlation (Pearson's R p-val >0.05) between stemness and all other scores across the cohort, immune activity and the GSEA-based invasiveness were strongly correlated (R 0.71, p-val 2.58e-134). Immune activity and invasiveness were also weakly, yet significantly, anticorrelated to the differentiation gene set enrichment score (R −0.27, p-val 5.52e-16).

The example experiments illustrate that iterative clustering provides a novel transcriptional stratification of thyroid tumours. The first observed split separates BRAF-mutated and BRAF-like tumours from all remaining samples (including healthy controls). Samples in this BRAF-like group show significantly increased immune activity, invasiveness and expression of stemness markers (p-val <4.65e-14). Thus, there is a possible link between BRAF mutations and invasiveness has been highly debated both within thyroid tumours specifically and in other malignancies. Thyroid differentiation markers, however, are significantly downregulated (p-val 2.22e-19).

Other significantly enriched pathways include general descriptors of thyroid cancer, papillary in particular, which is explained by the separation from normal tissue samples and Follicular carcionams. Rho GTPase binding, and α6β1-β4 integrin pathways are also enriched in BRAF-like tumours. The former regulates actin dynamics, cell migration and cell cycle progression. While not often considered in the context of thyroid carcinomas, its involvement in RET dysregulation has been observed specifically in thyroid epithelial cells. Integrin mediates extracellular-matrix adhesion and is under consideration as a target for cancer metastasis inhibition. Aberrant expression of this pathway has been described in dedifferentiated and anaplastic TC. In contrast, non BRAF-like samples are enriched for metabolic and biosynthesis pathways, relating to hormonal production.

The example experiments also illustrate that BRAF-like samples can be clustered by fusion type, immune profile and invasiveness. The hierarchy along the BRAF-like branch observes the separation of mTOR overexpressing samples from the rest of the population. This is a relatively small group of mostly BRAF-mutant samples, characterized by significant enrichment of mTOR and insulin growth factor 1 (IGF1) pathways; although no significant overexpression of IGF1 was observed in this cluster. While deregulation of PI3K/ATK/mTOR cascades is commonly associated with RAS-mutated samples, it has been described in BRAF-like classical-PTCs as well. More recently, IGF has been directly implicated in tumour progression and therapeutic resistance in thyroid malignancies. Interestingly, the remaining BRAF-like samples carry a transcriptional signal which overlaps with cluster 3. Other works focus on characterizing expression patterns of FTC, with and without PAX8-PPARγ fusion, and describe cluster 3 as being characterized by expression of ribosomal proteins and translation-associated genes, and a lack of PAX8-PPARγ lesion, but no mention of mTOR specifically has generally been made.

Further separating these two groups is the enrichment of Golgi-network, endoplasmic reticulum, vesicle and lysosome activity (e.g. the HOPS and SNARE complexes), DNA repair and telomere maintenance pathways in mTOR overexpressing samples. From these results, the present inventors suggest mTOR overexpression is characteristic of a specific subsection of BRAF^{V600E} PTCs, which are defined by a rich protein and vesicular production, abnormal lysosomal organization, and overactive 9 repair functions. Stemness is downregulated in this group (p-val 1.41e-2), an so is the incidence of lymphatic involvement (p-val 4.10e-9) and multifocality (p-val 3.80e-2) to a lesser degree, suggesting less chance of recurrence.

mTOR overexpressing samples further divides into two small classes at a next hierarchy level. The major driver of this separation seems to be quite clearly the level of immune infiltration, inflammatory response (p-val 3.95e-7, see FIG. 2d). IGF1 pathways are differentially enriched, and upregulated in the immune rich group (p-val <7.22e-3) and so is the expression of IGF1 (p-val=1.29e-7). In contrast, DNA, mismatch, RNA repair complexes, and processing pathways are all significantly enriched in the group with lower immune activity.

The vast majority of samples in the larger BRAF-like A are PTC from TCGA (83%) and they divide in 10 subclusters. Here, BRAF^{V660E}-positive samples intermix with those carrying alternative mutations in a common phenotype termed BRAF-like, but they are not distributed uniformly across these clusters. BRAF-like altFUS IMMhigh and BRAF-like altFUS host significantly more RET, NTRK1-3 and ALK fusions (p-val <3.89e-03), known oncogenes in PTC with therapeutic implications.

Aside from regulation of ion and transmembrane transport pathways, integrin, vascular endothelial growth factor (VEGF) and chemokine receptors signalling pathways (CXCR2, CXCR3 and CXCR4 in particular) are enriched. Differences in immune profiles are driving the separation of these two classes, with BRAF-like altFUS IMMhigh having a considerable enrichment of immune and inflammation-related pathways and higher invasiveness score (p-val 2.61e-9), suggesting these may be a subset of particularly aggressive tumours, prone to metastases, and possibly a priority target for immunotherapy.

BRAF AKT+ IMMhigh has similar levels of immune activity and invasiveness as BRAF-like altFUS IMMhigh but with a significant impoverishment of endothelial, VEGF and IGF1 pathways and upregulation of lysosomal and BLOC complex pathways. The example experiments observed a significant enrichment of AKT related pathways in this group, particularly in the presence of mTOR inhibition. These results are maintained when comparing BRAF AKT+IMMhigh with the remaining BRAF-like samples. Chemokine and interleukine-related pathways were also significantly up, while AKT activation through PI3K was downregulated. This characteristic upregulation of AKT by means alternative to PI3K and mTOR, while surprising, is not novel in the context of cancer.

At odds with their cohort-wise correlation, the example experiments observed enrichment of invasiveness-related gene sets (p-val 1.6e-11) with reduced immune activity (p-val 6.00e-3) in two classes, BRAF INVASIVE TCFB+ and BRAF INVASIVE TallCell. The stemness score is also significantly upregulated (p-val 5.85e-7) with respect to the remaining samples within the BRAF-like group.

BRAF INVASIVE TCFB+ showed significant enrichment of many gene sets downregulated in thyroid carcinoma and mTOR pathway, while thyroid hormone generation and response sets are enriched. Transforming growth factor beta (TGF-β) and sonic hedgehog (SHH) signalling pathways, known players in tumour growth and proliferation, are also upregulated; the former in particular is sensitive to thyroid hormone regulation.

While also overexpressing TCF-β pathways BRAF INVASIVE TallCell lacks the downregulation of most TCA characteristic gene sets. Other than that, samples in this group are enriched significantly for integrin, but impoverished for VEGFR and, counterintuitively, peroxisome proliferator-activated receptor (PPAR) signalling pathways; which have been associated with thyroid neoplasms aggressiveness. Furthermore, this group is characterized by a significant population of tall-cell PTC (19%), a variant known for its aggressiveness and poor prognostic viability. No samples from this group had a history of radiation exposure (p-val 1.43e-41).

A sizeable tall-cell population can also be found in BRAF REPAIR TallCell (22%); which is characterized by significant enrichment of DNA repair genes, synthesis, replication and recombination. Both tall cell groups have among the highest stemness scores, together with a third group, BRAF ESR- (p-val 8.17e-6 when taken together) characterized by significant overexpression of TCF-β pathways, PI3K in ERBB2 and ERBB4, and downregulation of catabolic processes and estrogen receptor ESR1 (ER-α) targets, commonly associated with poor prognosis. This is in contrast to BRAF-like ESR+ where several Ribonuclease, polymerase, excision repair, metabolic and catabolic pathways are upregulated, together with ESR1 targets. Metastatic incidence is the highest among all BRAF-like subtypes (4%) although not significant; previous studies found no correlation between ESR1 protein expression and invasiveness in PTC.

BRAF PI3K+ is enriched for metabolic pathways, amino acid biosynthesis, and more importantly, mTOR, ERBB, and PI3K activation and regulation. This group has the highest ratio of samples with a history of radiation exposure (6%) although the difference is not significant when compared to its sibling classes. BRAF VGF+ is defined by significant enrichment of nitrogen metabolism, VGEFR and NOTCH signalling pathways. NOTCH signalling might explain the observed upregulation of mixed cell differentiation.

The example experiments also illustrate that healthy normals can be categorized by their metabolic signature. The presence or absence of a BRAF-like expression profile acts as the prime driver in thyroid tissues stratification. All samples lacking this transcriptional signature can thus be grouped into a single class at first, non-BRAF-like. At the next level of the hierarchy the first division separates healthy normal tissue samples Normal Healthy from everything else, including most adjacent normals. This group is made almost exclusively of data from GTEx, except a handful of FTC-adjacent normal samples, and then splits into two subtypes. While the example experiments did not identify a significant difference in immune infiltration and stemness between the two, it was found that Normal Healthy METABOLIC is characterized by significant enrichment of numerous metabolic, biosynthesis and transport pathways, when compared to Normal Healthy HORMONE; which instead is overexpressing RNA polymerase II binding, development and differentiation gene sets for many different tissues, neural system in particular, and neurotransmitters activity. Hormone genes sets are also up in the latter given the role of thyroid hormones in the neural system development and growth.

The example experiments also illustrate that tumour-adjacent normals and RAS-like tumours share transcriptional similarities, and can be then split by histology and RAS pathway regulation levels. The remaining samples are found in RAS-like, which then splits into 8 separate classes, three of which are comprised of a majority of adjacent normal tissue samples matched to various tumours in the cohort. While a certain degree of mixing is observed across these three groups, Normal FAdj contains a significant majority of normals from follicular TC, follicular variant TC or follicular adenomas, while Normal PAdj is mostly made up of adjacent normal tissue taken from patients affected by papillary TC. Normal Adj IMMhigh is a mixture of normal tissues with different histology, brought together by an exceptional immune activity and invasiveness score. The present inventors hypothesize that these samples match particularly aggressive tumours, and clearly show that the effects of tumour infiltration extend well beyond the perimeter of the neoplastic population.

RAS-like tumour subtypes can then be organized into 5 separate classes. RAS FVPTC is the most populous group and contains a significant proportion of RAS-mutated samples, mostly NRAS when labelled, and follicular variant PTC. Its composition is reflected in the gene sets enrichment analysis. Indeed, this class shows significant upregulation of PTC gene sets, RAS pathways, specifically KRAS and seems to be impoverished for FTC specific gene sets, mTOR and IGF1-related gene sets instead. Mixed development and differentiation sets are also downregulated, while metabolic and catabolic sets are up. Mitogen-activated protein kinase MAPK is also downregulated.

Samples in RAS FTC have a similar transcriptional profile. These are also mostly RAS mutated samples, specifically both HRAS and NRAS, but the population of FTC represents the majority in this group, although many FV PTC are also present. Endoplasmic-reticulum associated protein degradation (ERAD), response to parathyroid hormone, VEGFR, KRAS pathways are upregulated and so are, to a lesser degree, mTOR and PI3K. Downregulation of ESR1 targets and MAPKKK activity was significant. All of these results were maintained when comparing this cluster with RAS FVPTC specifically, pointing at a stronger RAS pathway transcriptional signature in these samples.

The remaining classes were mostly comprised of samples with mutations alternative to RAS. RAS-like DICER1, is a small class, it contains all DICER1 mutated samples reported in the cohort. It showed significant enrichment for SHH, NOTCH, PI3K cascade pathways, epithelial differentiation and adhesion. In turn, PTC-associated genes, metabolic pathways, biosynthesis programs, but most importantly RAS and mTOR pathways were significantly under expressed when compared to all other tumour groups within this family. RAS-like OTHER, on the other hand, harbours EIF1AX mutations. NOTCH signalling regulation, SSH and FTC-related gene sets were all significantly up, while PTC markers, RAS and MAPK pathways were downregulated.

Also observed was the formation of a cluster of ESRRα-overexpressing tumours, RAS-like ESSRA+. Upregulation of estrogen-related receptor alpha has been described in what has been termed as Non-BRAF Non-RAS (NBNR) thyroid tumours and co-regulates mitochondrial activity in tandem with thyroid hormone receptor β1. Gene sets enrichment analysis also highlighted positive regulation of mTOR, response to thyroid hormone and metabolic pathways, and significant downregulation of RAS targets, SSH and integrin pathways, in addition to PTC marker genes.

The example experiments also illustrate that thyroid adenomas lack hallmark oncological expression signatures. When applying a recursive clustering search on the cohort, follicular adenomas were found intermixed with other RAS-like follicular and follicular variant papillary carcinomas across different clusters. This result is telling of the transcriptional variability among benign neoplasms and which outweighs their characteristic low aggressiveness when compared to other tumour types. The present inventors thus identified and investigated the specific gene expression profile of benign thyroid tumours when compared against all thyroid tumours; and RAS-like tumours specifically. The example experiments identified the characteristic transcriptional patterns of benign tumours when compared to their closest malignant counterparts, non-BRAF-like tumours. The latter are significantly enriched for thyroid cancer marker genes, and specifically KRAS and MAPK. On the other hand, benign nodules overexpress mTOR pathway genes, MET, RET, IGF1 and insulin pathways, but also sphingosine-1-phosphate (S1P) signalling, which is involved in cell proliferation. Deacetylase, which downregulates accessibility to transcription factors (TF) is the highest-ranking pathway in the gene sets enrichment analysis for benign tumours, and acetyltransferase is also significantly upregulated. On the other hand, it was observed that a strong downregulation of exploration and vesicle-related pathways, thyroid and parathyroid binding and response to starvation and calnexin cycle, which regulates 14 protein folding. These results were maintained, when compared with the whole set of tumours within the cohort, with expected rearrangements in the ranking of thyroid marker gene sets, mTOR and KRAS pathways, although significance was still observed.

From these results, the experiments suggest that nodules are expressing most of the transcriptional hallmarks common to malignant thyroid tumours but to a lower extent, except for the mTOR pathway, but they showed a remarkably diminished level of invasiveness. Indeed, while this group had a similar immune activity score as other tumours, the invasiveness score confirms the above. Stemness is also elevated significantly in this group against all others. Particularly interesting is the overexpression of deacetylase, which seems to suggest these entities are less prone to (TF) activation. 6,079 genes were differentially expressed between the two groups, subset 460 upregulated and 941 downregulated genes, and were used to build gene sets that optimize the separation between benign and RAS-like malignant neoplasms (p-val 3.59e-12 and 5.05e-09 respectively for up- and downregulation). While gene sets containing markers of follicular adenomas are generally known, it was found that they were insufficient to separate RAS-like tumours from benign nodules. Similarly, 53 upregulated and 208 downregulated genes out of 1,515 were sufficient to separate benign tumours against all malignant neoplasms with optimal p-values (p-val 2.38e-20 and 2.49e-20 respectively). High-accuracy classification of benign and malignant samples by transcriptome is thus only possible, as proved by the high significance of the results of the example experiments.

The example experiments also illustrate that tumour-adjacent normal tissue carries high energy pathways signals. The comparison of benign tumours against all other groups, BRAF-like tumours and normals included, confirms how thyroid neoplasms sit on a spectrum of aggressiveness and tumorigenicity; with healthy normal tissues and BRAF-like tumours at the ends, tumour-adjacent normals and RAS-like malignant tumours are in-between and carry similar tumoral profiles, albeit less aggressive. The separation between healthy normal tissues and tumour-adjacent ones is evident, and thus the present inventors chose to characterize it more in detail, following a similar approach used to classify benign and malignant tumours.

With the exception of a single gene set of markers of poorly differentiated TC, upregulated in tumour-adjacent samples, differentially expressed gene sets are not thyroid-specific, supporting that the method 2000 can be applied to all tissue types.

Metabolic pathways, specifically of sphingolipid, which are involved in a plethora of signalling cascades including apoptosis, autophagy, proliferation and stress response, glycan and steroid biosynthesis were upregulated in tumour-adjacent samples. Along with the peroxisome pathway and GTPase activity, Vesicle production and transport pathways, together to respond to endoplasmic reticulum stress.

Interestingly, the example experiments also observed significant upregulation of mismatch repair, transcription-coupled nucleotide excision repair, RNA degradation, stabilization and a number of protein folding and production pathways in this group. This has an impact on the presence of contiguous neoplasm. These results draw the picture of a biological system in high alert, with dysregulation of energy production, signalling and active stress response pathways. The overactive repair pathways are also a telltale sign of tumour adjacency. On the other hand, RhoA regulatory pathway, which modulates cell shape and locomotion, VEGF, integrin, notch signalling, and histone deacetylation, which hampers transcriptional factors activity, were all significantly downregulated. Healthy normals also seemed to be characterized by significant enrichment of ion channels and neurotransmitter transport, and cGMP biosynthesis and metabolism, also a regulator of ion channels function and cell apoptosis. Both apoptotic cell clearance and cell maturation were also elevated in this group of samples. As above, of 10,608 differentially expressed genes, only 115 upregulated and 964 downregulated in tumour-adjacent normal samples were selected to build a gene set able to distinguish them from healthy normal samples with an optimal degree of separation (p-val 5.20e-21 for both upregulated and downregulated samples respectively).

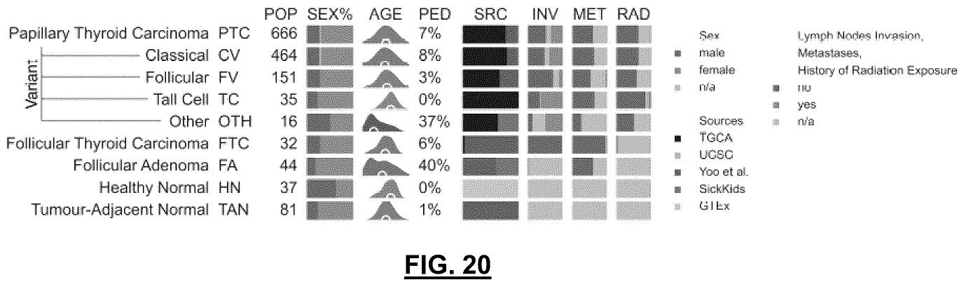

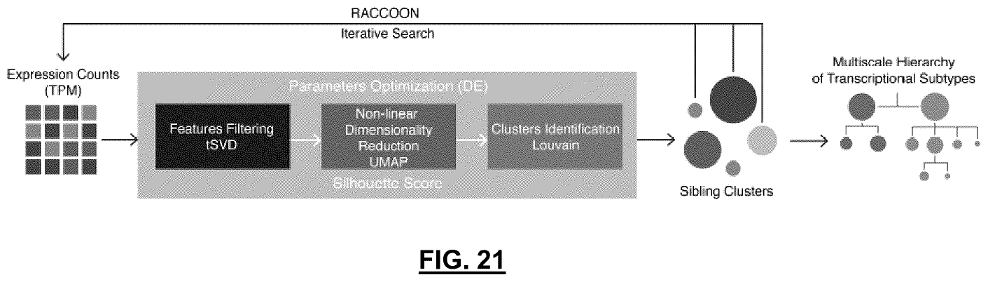

FIG. 20 illustrates a summary of clinical information from the mixed-source dataset used in the example experiments; including population size, sex ratio, age, percentage of pediatric samples (<=18 y.o.), source data sets, radiation history and presence of metastases. Samples are grouped by diagnosis shared by the presenting institution, tumour subtype and/or variant. FIG. 21 illustrates a flowchart showing the main steps of the multi-scale iterative clustering approach, as described herein. Starting from the expression tables, a clusters identification workflow is applied with parameters 17 optimized. This approach is repeated iteratively for each newly identified cluster until given exit conditions on population or optimization score are met. The final result is a hierarchy of transcriptionally-driven clusters.

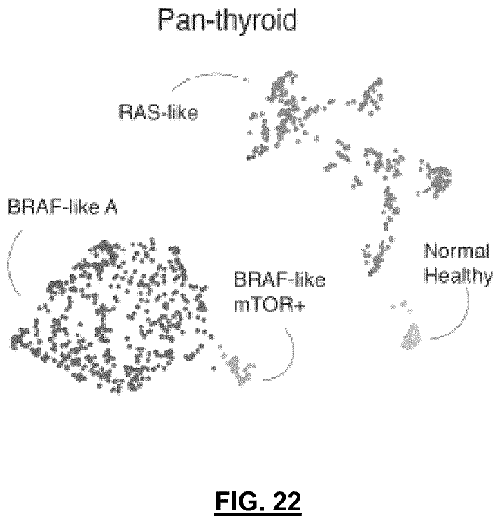

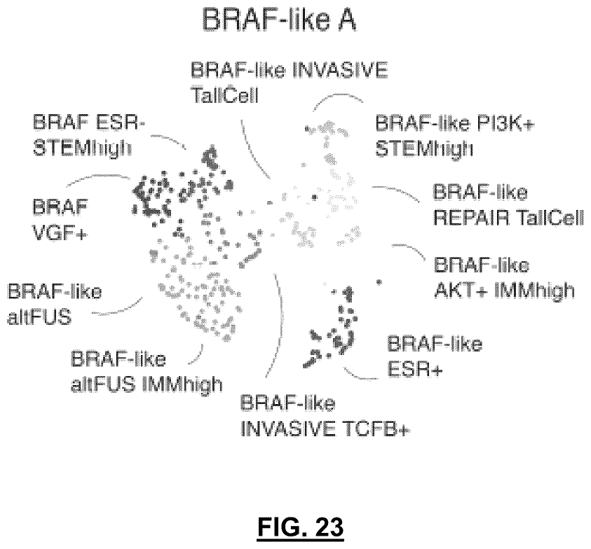

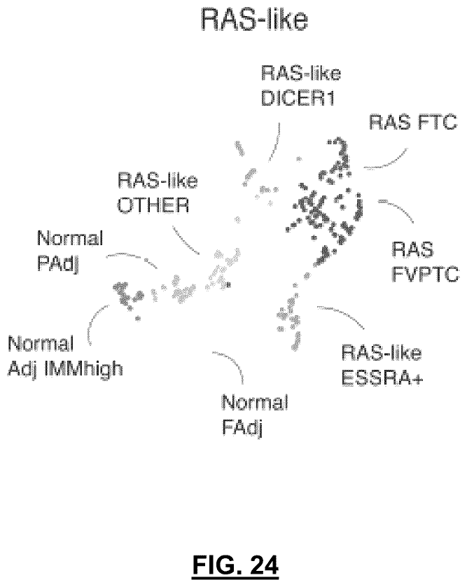

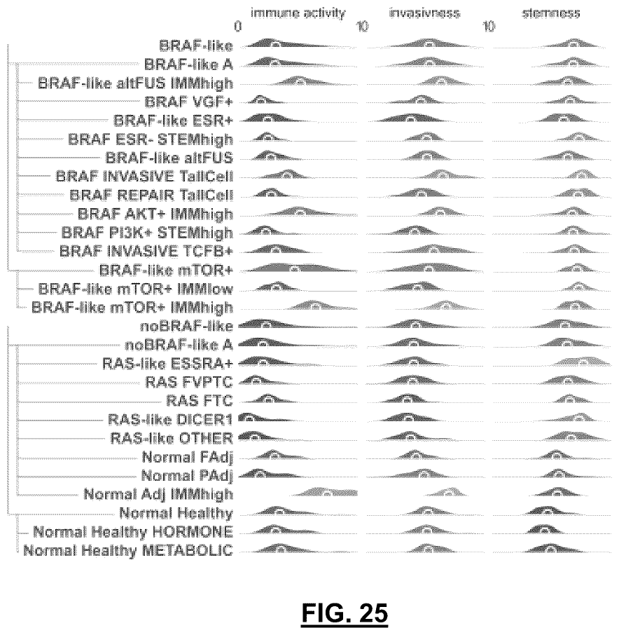

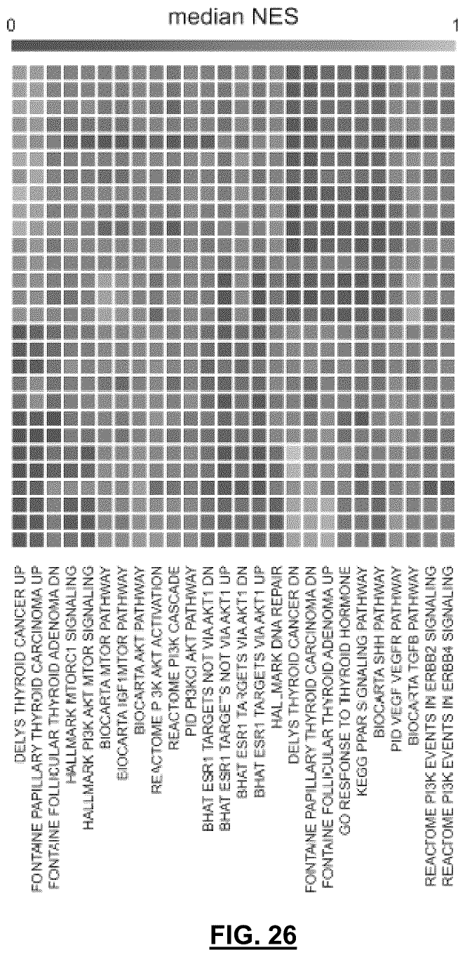

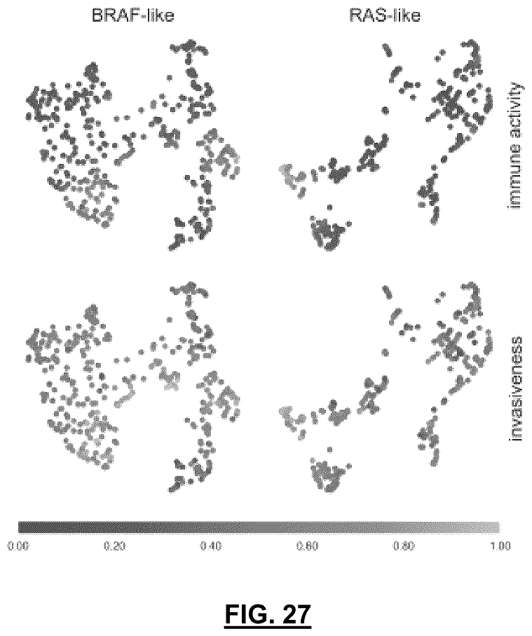

FIGS. 22 to 24 show two-dimensional UMAP representations of the full thyroid cohort (FIG. 22), the BRAF-like A subclass (FIG. 23), and the RAS-like subclass (FIG. 24), where relevant subclusters are shaded accordingly. FIG. 25 shows the first three layers of the thyroid samples cluster hierarchy. Distributions of the stemness, immune activity and invasiveness scores are shown for each cluster. The median value is shown as a white circle. FIG. 26 shows a heatmap illustrating the median normalized enrichment score of a curated subset of gene sets. FIG. 27 shows UMAP projections of BRAF-like and RAS-like samples shaded as a function of their immune and invasiveness scores.

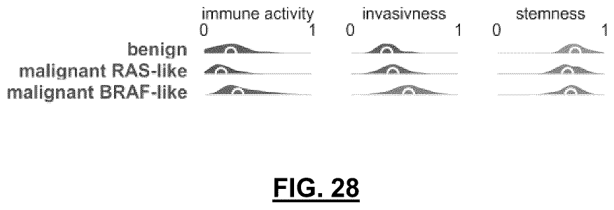

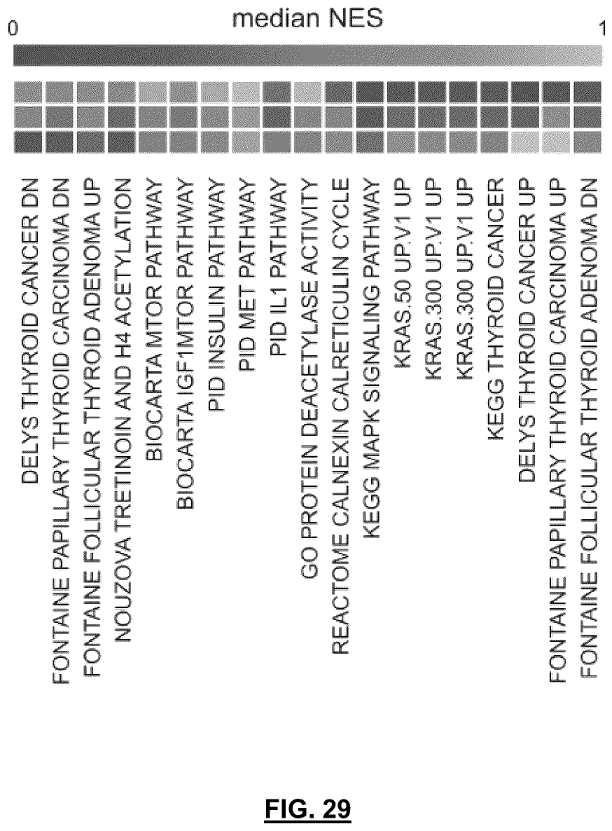

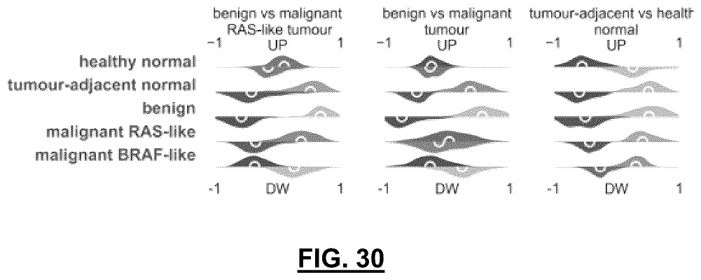

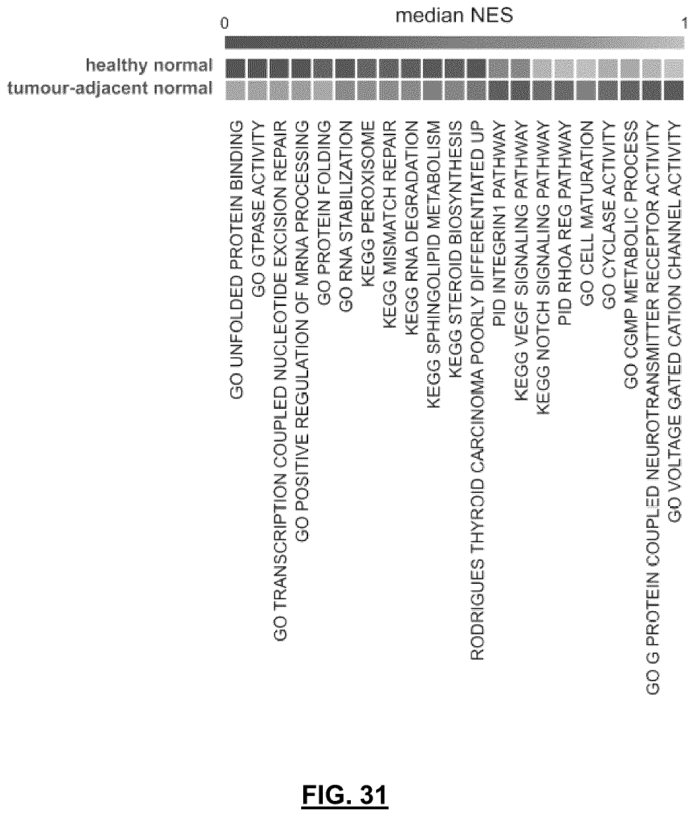

FIG. 28 shows distributions of the stemness, immune activity and invasiveness scores stratified by benign nodules or malignant. FIG. 29 shows median normalized enrichment score for a set of representative gene sets across benign and malignant tumours. FIG. 30 shows normalized enrichment score distributions for a group of in-house defined gene sets. These were designed to optimize the separation of benign vs malignant tumours (left), benign vs malignant RAS-like tumours (center), and healthy normal vs tumour-adjacent normal samples (right). FIG. 31 shows median normalized enrichment score for a set of representative gene sets across healthy and tumour-adjacent normal samples.

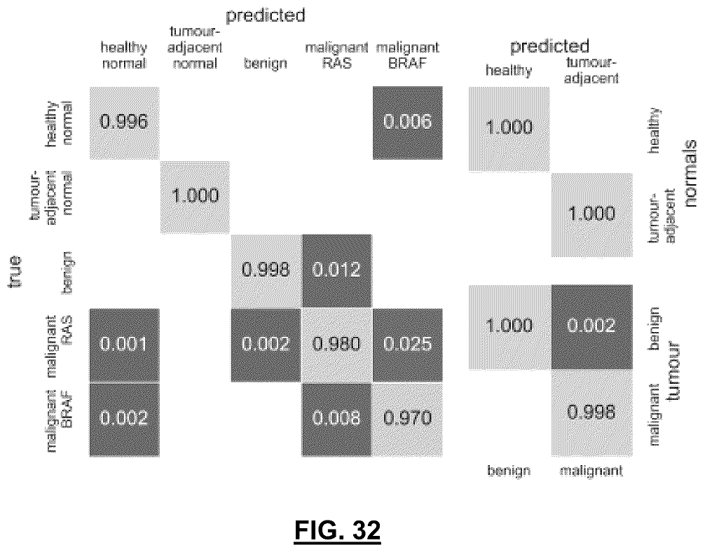

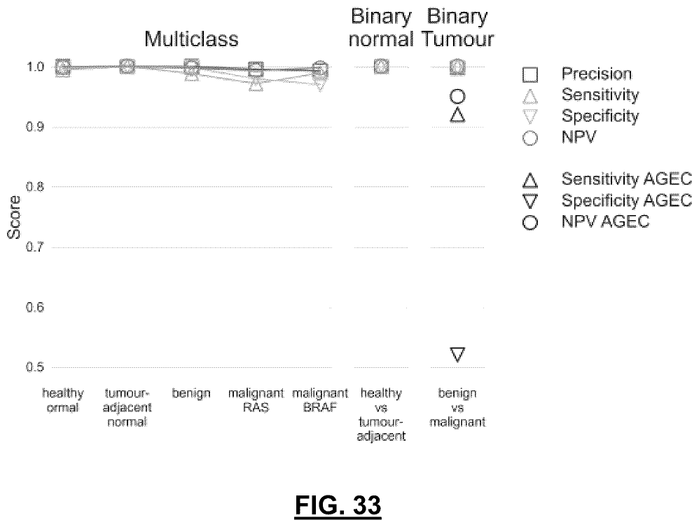

FIG. 32 shows a confusion matrix of the multiclass GBoost classifier (left), healthy versus tumour-adjacent normals binary classifier (top right), and benign versus malignant tumour binary classifier (bottom right). The matrix shows the percentage of samples correctly or incorrectly predicted against their true label. The values are obtained as the average of the randomized 5×5-fold validation. FIG. 33 shows performance scores for each of the classes predicted by the three GBoost models; along with the results reported for the Afirma™ Gene Expression Classifier (AGEC).

Although the invention has been described with reference to certain specific embodiments, various other aspects, advantages and modifications thereof will be apparent to those skilled in the art without departing from the spirit and scope of the invention as outlined in the claims appended hereto.

1. A computer-implemented method for classification of cancer from gene expression input data, the method comprising:
receiving a training dataset comprising nucleic acid data points from one or more samples;
identifying classes in the training dataset comprising performing recursive clustering by, at each successive iteration, performing a search to identify clusters based on similarity, wherein each class of the classes is associated with a tumor;
training a machine learning model to associate the nucleic acid data points of the training dataset with the identified classes;
receiving the gene expression input data for classification;
classifying, using the trained machine learning model, the gene expression input data as one or more of the identified classes; and
outputting the classification of the gene expression input data.

2. The method of claim 1, further comprising, prior to identifying classes, performing removal of low information features by ranking features by their respective variance and removing features whose variance is below a pre-determined cut-off.

3. (canceled)

4. The method of claim 2, wherein the variance is determined using Shannon's entropy.

5. The method of claim 1, further comprising, prior to identifying the classes, performing non-linear dimensionality reduction by performing Uniform Manifold Approximation and Projection.

6. (canceled)

7. The method of claim 1, wherein optimizing the input dataset comprises determining a score representative of a quality of the identified clusters by determining a ratio between cohesion of the clusters and separation of the clusters.

8. The method of claim 1, further comprising performing a grid search to determine optimized parameters for identifying the clusters.

9. The method of claim 8, wherein determining the optimized parameters is repeated for each iteration using only data included in a given cluster to identify hierarchical subclusters.

10. The method of claim 8, wherein, once clusters and subclusters are identified, each cluster is successively selected and parameters are optimized internally for such cluster, further iterations of optimization are performed following a branch stemming from such cluster.

11. The method of claim 7, wherein performing recursive clustering comprises:
iteratively identifying clusters with different parameters for each iteration;
evaluating each identification of clusters using an internal validation score; and
selecting the identification of clusters with the highest score.

12. The method of claim 11, further comprising determining complexity of a hierarchical branch stemming from each cluster based on a number of offspring hierarchical nodes.

13. The method of claim 1, wherein the trained machine learning model outputs membership probability to one or more of the identified classes.

14. The method of claim 1, wherein the trained machine learning model comprises an ensemble of convolutional neural networks, each convolutional neural network comprises one or more one-dimensional convolutional layers, followed by one or more fully connected layers with dropout, and followed by a fully connected layer.

15. (canceled)

16. The method of claim 1, wherein the trained machine learning model is multiclass such that the gene expression input data can be assigned to more than one class.

17. The method of claim 1, further comprising performing agglomerative clustering on log 2 normalized expressions of the gene expression input data prior to classification.

18. The method of claim 1, wherein the input dataset comprises genes as features and expression counts of such genes as values.

19. The method of claim 1, wherein the reference dataset comprises gene expression from healthy tissue.

20. The method of claim 1, further comprising identifying gene expression outliers in the gene expression input data prior to classification, comprising comparing gene expression for the input data against gene expression distributions of reference tumours and normal tissues.

21. The method of claim 1, wherein each of the classes are associated with a type of sarcoma tumor.

22. The method of claim 21, wherein the classes are associated with one or more of osteosarcoma, leiomyosarcoma, fusion-positive rhabdomyosarcoma, fusion-negative rhabdomyosarcoma, synovial sarcoma, and Ewing sarcoma.

23. A system for classification of cancer from gene expression input data, the system comprising:
an input module to receive a training dataset comprising nucleic acid data points from one or more samples and receive the gene expression input data for classification;
an optimization module to identify classes in the training dataset comprising performing recursive clustering by, at each successive iteration, performing a search to identify clusters based on similarity, wherein each class of the classes is associated with a tumor;
a training module to train a machine learning algorithm to associate the nucleic acid datapoints of the training dataset with the identified classes;
a classification module to classify, using the trained machine learning model, the gene expression input data as one or more of the identified classes; and
an output module to output the classification of the gene expression input data.

24-91. (canceled)
